(19) United States
(12) Patent Application Publication
(10) Pub. No.: US 2013/0344490 A1
(43) Pub. Date: Dec. 26, 2013

(54) NEOPLASTIC CELLS GROWN ON DECELLULARIZED BIOMATRIX

(71) Applicant: Min Peter Kim, Houston, TX (US)

(72) Inventor: Min Peter Kim, Houston, TX (US)

(21) Appl. No.: 13/871,202

(22) Filed: Apr. 26, 2013

Related U.S. Application Data
(60) Provisional application No. 61/639,435, filed on Apr. 27, 2012.
(60) Provisional application No. 61/755,867, filed on Jan. 23, 2013.

(51) Int. Cl. G01N 33/50

(52) U.S. Cl. CPC G01N 33/5044
USPC 435/6.12; 435/395; 435/366; 435/347; 435/29; 435/39; 435/289.1

(57) ABSTRACT

Some aspects of this disclosure provide tissue constructs comprising a decellularized biomatrix and a neoplastic cell cultured within the biomatrix, as well as methods, reagents, and bioreactors for generating and using such tissue constructs. Tissue constructs as provided herein resemble clinically presenting tumors more closely than conventional in vitro and in vivo tumor models in various aspects, and can be used, for example, as tumor models for research and for the identification of anti-cancer agents.

### RELATED APPLICATIONS

This application claims priority under 35 U.S.C. §119(e) to U.S. provisional patent application No. 61/639,435, filed Apr. 27, 2012, and U.S. provisional application No. 61/755,867, filed Jan. 23, 2013, both entitled “Neoplastic Cells Grown On Decellularized Biomatrix,” the entire contents of each of which are incorporated herein by reference.

### BACKGROUND

Cancer is the second leading cause of death in the United States. The understanding of tumor biology and the development of new and improved treatments of solid tumors is hampered by the limitations of in vitro and in vivo disease models in use today, which translate poorly into clinical practice because of their lack of concordance with tumors in human patients. Tumor models that more closely reflect the conditions in patients are required.

### SUMMARY

Some aspects of this disclosure are based on the recognition that one reason for the lack of concordance between current tumor models and tumors presented in the clinic is the shortcomings of current model systems in modeling the effect of the interaction of the tumor cells with surrounding structures in vivo. In a subject, a neoplastic cell, for example, a cancer cell, interacts with the extracellular matrix and other cells of the host tissue, and these interactions are either not possible or severely limited in most current tumor models. For example, a common assay for determining the metastasizing potential of tumor cells measures the cells' ability to transgress an artificial barrier, which a tumor cell will never encounter in the in vivo environment of a human patient. Similarly, synthetic two- or three-dimensional culture surfaces used for the culture of cancer cells do not truly mimic human conditions. While in vivo cancer models theoretically allow the study of interactions of neoplastic cells with host tissues by growing human cancer cells in an animal host (e.g., a mouse or a rat), there are severe limitations on the translation of data obtained from such models to the clinic, because the interaction of human neoplastic cells with animal tissue is not representative of the interaction with human tissue, and also because the host animal is typically immunocompromised, while neoplastic cells that give rise to cancer in humans are confronted with the responses of the human immune system.

Some aspects of this disclosure relate to the surprising discoveries that (i) the extracellular matrix (ECM) of the host tissue plays an important role in allowing solid tumors, for example, lung cancer tumors, to grow and to acquire and maintain their shape and organization; (ii) tissue constructs useful as tumor models can be engineered by growing neoplastic cells on decellularized biomatrix; (iii) suitable decellularized biomatrix can be obtained from a tissue or organ from a subject via decellularization; (iv) tissue constructs comprising neoplastic cells cultured within decellularized biomatrix more closely resemble endogenous tumors formed in a subject than currently used in vitro or in vivo 2D and 3D model systems, both in structural and functional aspects; and (v) tissue constructs generated by growing neoplastic cells on decellularized biomatrix can be employed for researching tumor biology and to identify anti-cancer agents.

Some aspects of this disclosure provide tissue constructs. In some embodiments, the tissue construct comprises a decellularized biomatrix and a neoplastic cell cultured within the decellularized biomatrix. In some embodiments, the tissue construct comprises a tumor nodule. In some embodiments, the neoplastic cell is not native to the decellularized biomatrix. In some embodiments, the neoplastic cell is from a different species than the decellularized biomatrix. In some embodiments, the decellularized biomatrix is derived from a healthy tissue or organ obtained from a subject. In some embodiments, the tissue construct comprises a perfusable vasculature. In some embodiments, the decellularized biomatrix comprises lung biomatrix. In some embodiments, the decellularized biomatrix comprises rat or mouse biomatrix. In some embodiments, the neoplastic cell is a human cell. In some embodiments, the neoplastic cell is a tumor or cancer cell. In some embodiments, the tissue construct further comprises a non-neoplastic cell.

Some aspects of this disclosure provide methods for preparing a tissue construct. In some embodiments, the method comprises providing a decellularized biomatrix and contacting the decellularized biomatrix with a neoplastic cell under conditions suitable for the neoplastic cell to grow within the decellularized biomatrix. In some embodiments, the conditions are suitable for the cell to form a tumor nodule within the decellularized biomatrix. In some embodiments, the decellularized biomatrix is from a different species than the neoplastic cell. In some embodiments, the decellularized biomatrix comprises rat or mouse biomatrix. In some embodiments, the neoplastic cell is a human cell. In some embodiments, the decellularized biomatrix comprises decellularized lung biomatrix. In some embodiments, the lung biomatrix comprises a trachea, and wherein the contacting comprises infusing a medium containing the neoplastic cell into the trachea. In some embodiments, the method comprises perfusing the decellularized biomatrix contacted with the neoplastic cell with a culture medium. In some embodiments, the method further comprises contacting the decellularized biomatrix with a non-neoplastic cell. In some embodiments, the method further comprises analyzing the tissue construct. In some embodiments, the analyzing comprises observing a tumor nodule, observing growth of a tumor nodule, quantifying a number of tumor nodules, assaying expression of a gene product associated with neoplasia, assaying cell survival or cell death, assaying metastatic potential, and/or assaying a signaling factor associated with neoplasia.

Some aspects of this disclosure provide methods of identifying an anti-cancer agent. In some embodiments, the method comprises (a) contacting a tissue construct provided herein with a candidate agent; (b) assessing a biomarker associated with cancer in the tissue construct contacted with the candidate agent; and (c) comparing the assessed biomarker of (b) with a reference value. In some embodiments, if the biomarker associated with cancer is absent or diminished in the tissue construct contacted with the candidate agent as compared to the reference value, then the candidate agent is identified as an anti-cancer agent. In some embodiments, the biomarker assessed in (b) comprises cell proliferation, cell survival, tumor formation, tumor number, tumor growth, tumor volume, tumor phenotype, tumor nodule formation, tumor nodule number, tumor nodule growth, tumor nodule structure, tumor nodule volume, tumor nodule phenotype, expression of a gene product, expression of an oncogene, repression of a tumor suppressor, presence or abundance of neoplastic cells in a perfusion efflux fluid, expression of mesenchymal markers by cells present in a perfusion fluid, and/or a metastatic activity of cells present in a perfusion fluid. In some embodiments, the reference value is a value observed or expected in a tissue construct not contacted with a candidate agent. In some embodiments, the candidate agent is a small molecule compound, a nanoparticle, a nucleic acid, an RNAi agent, a protein, or an antibody or antibody fragment. In some embodiments, the method is used to screen a library of candidate agents.

Some aspects of this invention provide a bioreactor for growing perfusable tissue constructs, for example, tissue constructs comprising neoplastic cells and decellularized biomatrix as provided herein. In some embodiments, the bioreactor comprises a decellularized biomatrix having a vascular space and an epithelial space; a perfusion influx connected to the vascular space; a perfusion efflux connected to the vascular space; a culture media influx connected to the epithelial space; and a neoplastic cell growing within the decellularized biomatrix and contacted with the culture media. In some embodiments, the decellularized biomatrix comprises an artery and a vein. In some embodiments, the perfusion influx is connected to the artery and/or the perfusion efflux is connected to the vein. In some embodiments, the decellularized biomatrix is a lung decellularized biomatrix. In some embodiments, the bioreactor does not comprise a ventilation loop. In some embodiments, the lung decellularized biomatrix comprises a trachea and the culture media influx is connected to the trachea. In some embodiments, the neoplastic cell is from a different species than the decellularized biomatrix.

Some aspects of this invention provide a metastatic tumor model. In some embodiments, the metastatic tumor model comprises a decellularized biomatrix, which comprises a primary interstitial space and a neoplastic cell within the primary interstitial space; a secondary interstitial space that does not comprise a neoplastic cell; a barrier to cell migration that separates the primary and the secondary interstitial space; and a vascular space shared by the primary interstitial space and the secondary interstitial space. In some embodiments, the shared vascular space comprises a perfusion medium. In some embodiments, the decellularized biomatrix is derived from a healthy tissue or organ obtained from a subject. In some embodiments, the primary and the secondary interstitial space are comprised in a biomatrix derived from a single tissue or organ, or derived from the same type of tissue or organ. In some embodiments, the decellularized biomatrix comprises lung biomatrix. In some embodiments, the decellularized biomatrix is derived from a single lung and comprises a plurality of bronchi and/or a plurality of lobes, and wherein the primary interstitial space is comprised in one bronchus or lobe and the secondary interstitial space is comprised in a different bronchus or lobe. In some embodiments, the primary interstitial space is seeded with a neoplastic cell, and wherein the secondary interstitial space is not seeded with a neoplastic cell.

Some aspects of this invention provide a method for cultivating neoplastic cells. In some embodiments, the method comprises providing a decellularized biomatrix that comprises a primary interstitial space; a secondary interstitial space, wherein the secondary interstitial space does not comprise a neoplastic cell; a barrier to cell migration that separates the primary and the secondary interstitial space; and a vascular space shared by the primary interstitial space and the secondary interstitial space, wherein the vascular space comprises a perfusion medium; and contacting the primary interstitial space of the biomatrix with a neoplastic cell under conditions suitable for the neoplastic cell to grow within the decellularized biomatrix. In some embodiments, the decellularized biomatrix comprises decellularized lung biomatrix. In some embodiments, the lung biomatrix comprises a trachea, and wherein the contacting of the primary interstitial space with a neoplastic cell comprises infusing a medium containing the neoplastic cell into the trachea. In some embodiments, the decellularized lung biomatrix comprises a plurality of bronchi and/or a plurality of lobes, and wherein the primary interstitial space is comprised in one bronchus or lobe and the secondary interstitial space is comprised in a different bronchus or lobe. In some embodiments, the primary interstitial space is separated from the secondary interstitial space by an airway ligation or a tracheal ligation. In some embodiments, the method comprises perfusing the decellularized biomatrix contacted with the neoplastic cell with a culture medium. In some embodiments, the method further comprises culturing the neoplastic cell within the biomatrix for a time period sufficient for the formation of circulating cells. In some embodiments, the method further comprises culturing the neoplastic cell within the biomatrix for a time period sufficient for a neoplastic cell to invade the secondary interstitial space or for the formation of a tumor nodule in the secondary interstitial space. In some embodiments, the method further comprises isolating and/or analyzing a cell or tissue obtained from the biomatrix.

Some aspects of this invention provide a method of identifying an anti-metastatic agent. In some embodiments, the method comprises (a) contacting a metastatic tumor model described herein with a candidate agent; (b) assessing a biomarker associated with metastasis in the tissue construct contacted with the candidate agent; and (c) comparing the assessed biomarker of (b) with a reference value. In some embodiments, if the biomarker associated with metastasis is absent or diminished in the tissue construct contacted with the candidate agent as compared to the reference value, then the candidate agent is identified as an anti-metastatic agent. Some aspects of this invention provide a metastatic tumor model. In some embodiments, the metastatic tumor model comprises a decellularized biomatrix, which comprises a primary interstitial space and a neoplastic cell within the primary interstitial space; a secondary interstitial space that does not comprise a neoplastic cell; a barrier to cell migration that separates the primary and the secondary interstitial space; and a vascular space shared by the primary interstitial space and the secondary interstitial space. In some embodiments, the biomarker assessed in (b) comprises presence or abundance of neoplastic cells in a perfusion fluid, expression of mesenchymal markers by cells present in a perfusion fluid, a metastatic activity of cells present in a perfusion fluid, presence or abundance of neoplastic cells in the secondary interstitial space, and/or the presence or abundance of tumor nodules in the secondary interstitial space. In some embodiments, the reference value is a value observed or expected in a tissue construct not contacted with a candidate agent. In some embodiments, the candidate agent is a small molecule compound, a nanoparticle, a nucleic acid, an RNAi agent, a protein, or an antibody or antibody fragment. In some embodiments, the method is used to screen a library of candidate agents.

Other advantages, features, and uses of the invention will be apparent from the detailed description of certain non-limiting embodiments; the drawings, which are schematic and not intended to be drawn to scale; and the claims.

### BRIEF DESCRIPTION OF THE DRAWINGS

FIG. 1. Decellularization unit and bioreactor. (A) Three customized decellularization chambers connected to a pump.(B) Four customized bioreactors inside an incubator connected to a pump and an oxygenator.

FIG. 2. Decellularized rat lung.(A) Freshly harvested intact native rat lung with heart block. (B) Hematoxylin and eosin staining of native rat lung showing cellularized alveoli with pneumocytes and endothelial cells. (C) Translucent acellular lung after sodium dodecyl sulfate and Triton-X perfusion through pulmonary artery. (D) Hematoxylin and eosin staining of acellular lung lobe showing absence of any cells. Lower panel: time line of decellularization process.

FIG. 3. DNA concentration of native and acellular lung. Equal amounts of tissue samples were taken for DNA extraction using Qiagen kit. DNA concentration was significantly reduced to less than 5% of native lung tissue.

FIG. 4. Upper panel: Exemplary bioreactor for growing cells on decellularized lung biomatrix. Cells are infused through the trachea and cultured in the interstitial space of the biomatrix, while media is perfused through the pulmonary artery. BM: basement membrane. Lower panel: A549 human lung cancer cell line grown on acellular rat lung matrix. (A) Lung matrix with A549 cells on day 11 showing tumor nodules. (B) Hematoxylin and eosin staining of left upper lobe on day 3 showing cells attached to matrix in airways, terminal bronchioles, alveolar ducts, and alveoli with no cells in vasculature. (C, D) Immunohistochemistry staining of lung seeded with A549 cells using Ki-67 (C) shows high proliferative index and using vimentin (D) shows presence of intermediate filaments.

FIG. 5. Lung matrix seeded with H460 cells. (A) Numerous tumor nodules on day 7 in lung scaffold. (B, C) Hematoxylin and eosin staining of lung section in low-power field (B) and high-power field (C) showing poorly differentiated non-small cell lung cancer with sheetlike growth along airways and alveoli.

FIG. 6. Lung matrix seeded with H1299 cells. (A) Numerous tumor nodules on day 7 in lung scaffold. (B, C) Hematoxylin and eosin staining of lung section in low-power field (B) and high-power field (C) showing very poorly differentiated non-small cell lung cancer cells in disorganized fashion.

FIG. 7. Comparison of several biomarkers observed in 2D culture of neoplastic cells and in 3D culture of the same cells on decellularized lung biomatrix.

FIG. 8. Comparison of Ki-67 and TUNEL staining in 2D and 3D culture after 15 days of culture.

FIG. 9. Schematic of perfused tissue construct. Human A549 lung cancer cells were infused into a decellularized lung biomatrix and cultured in the interstitial space of the biomatrix. The vascular space of the biomatrix was perfused through the pulmonary artery. Secreted factors, e.g., matrix metalloproteases (MMPs) are represented by circles in the vascular space. BM: basement membrane of the tissue construct.

FIG. 10. Comparison of MMP secretion in 2D neoplastic cell culture to MMP secretion in tissue constructs with or without neoplastic cells.

FIG. 11. Schematic of an exemplary experimental setup for assessing the effect of a drug, for example, an anti-cancer drug or a candidate agent, on cancer-associated biomarkers in tissue constructs comprising lung decellularized biomatrix and neoplastic cells.

FIG. 12. Exemplary tissue constructs at different days of culture. The left construct in each of the four panels (day 4, day, 8, day 11, day 14), was not treated, while the right construct was treated with Cisplatin.

FIG. 13. Tumor size and live tumor cells in treated and untreated lung tissue constructs. Tx: start of treatment, RUL: lobectomy of right upper lobe, RML: lobectomy of right middle lobe.

FIG. 14. H&E staining, Ki-67 staining, and TUNEL staining in treated and untreated lung tissue constructs.

FIG. 15. Circulating cells in perfusion fluid in treated and untreated lung tissue constructs.

FIG. 16. MMP secretion into perfusion fluid in treated and untreated lung tissue constructs.

FIG. 17. Comparison of lung biomatrix constructs comprising non-metastatic 393P lung adenocarcinoma cells to lung biomatrix constructs comprising metastatic 344SQ lung adenocarcinoma cells. No difference was observed in tumor nodule growth in both constructs but a higher number of circulating tumor cells was detected in constructs comprising 344SQ cells as compared to constructs comprising 393Pcells. The circulating cells exhibited a greater ability to migrate as compared to the cultured cells in a Boyden chamber assay.

FIG. 18. H&E staining, Ki-67 staining, and TUNEL staining in lung tissue constructs comprising non-metastatic 393P cells and metastatic 344SQ cells.

FIG. 19. Upper panel: Overview over epithelial-mesenchymal transition (EMT). Middle panel: Exemplary marker proteins that are regulated during EMT. Lower panel: comparison of exemplary marker protein expression in 393P and 344SQ cells.

FIG. 20. Comparison of EMT marker expression in 393P cells in 2D culture (“cultured”) to expression in cells recovered from perfusion fluid of tissue constructs comprising decellularized lung biomatrix and 393P cells (“circulating”).

FIG. 21. Comparison of EMT marker expression in 344SQ cells in 2D culture (“cultured”) to expression in cells recovered from perfusion fluid of tissue constructs comprising decellularized lung biomatrix and 344SQ cells (“circulating”).

FIG. 22. Comparison of EMT marker expression in 393P cells in 2D culture (“cultured”) to expression in cells recovered from the tissue or the perfusion fluid of tissue constructs comprising decellularized lung biomatrix and 393P cells (“tissue,” and “circulating,” respectively).

FIG. 23. Comparison of EMT marker expression in 344SQ cells in 2D culture (“cultured”) to expression in cells recovered from perfusion fluid of tissue constructs comprising decellularized lung biomatrix and 344SQ cells (“circulating”).

FIG. 24. Upper panel: Schematic of perfused tissue construct. Human A549 lung cancer cells were infused into a decellularized lung biomatrix and cultured in the interstitial space of the biomatrix. The vascular space of the biomatrix was perfused through the pulmonary artery. Circulating cells that have migrated across the basement membrane into the vascular space are represented by diamonds in the vascular space. BM: basement membrane of the tissue construct. Middle panel: Measurement of the number of circulating cells over a time period of 15 days. Lower panel: comparison of gene expression levels in cells residing in the interstitial space of the biomatrix and circulating cells.

FIG. 25. Measurements of tumor nodule size and number of circulating cells in tissue constructs seeded with NIH-H1299 cells.

FIG. 26. Upper panel: Metastatic tumor model. After one bronchus was tied off to prevent influx of cells, cancer cells were infused into the other bronchus of a decellularized lung biomatrix and cultured in the interstitial space of the bronchus, forming primary tumors. The vascular space of both bronchi was perfused through the pulmonary artery. Circulating cells that have migrated into the vascular space were able to transgress the basement membrane of the tied-off bronchus and to form secondary tumors in the interstitial space of the tied-off bronchus. Middle panel: histology of a primary tumor in the metastatic tumor model. Lower panel: number of circulating cells measured over a time period of 28 days in the metastatic model.

FIG. 27. Upper panel: Histology of secondary tumors and tumor nodules formed in the metastatic model. Serial lobectomy was performed on the tied-off bronchus, with removal of one bronchial lobe each at day 14, day 21, and day 28, respectively, and lobe histology was examined via H&E staining. Middle panel: histology detail of metastatic lesions on day 14 and 28. Lower panel: Quantification of tumor cell number per high power field (HPF) in metastatic lesions from day 14, 21, and 28, respectively.

### DETAILED DESCRIPTION

### Definitions

The term “tissue construct,” as used herein, refers to a composition comprising a substantially acellular matrix, for example, a decellularized biomatrix as described herein, and a cell growing within that matrix. A tissue construct typically represents a three-dimensional cell culture, in which the cell or cells adhere to the matrix and form a tissue or a tissue-like structure. The matrix comprised in a tissue construct typically functions as a substrate for the cell(s) to adhere to and to migrate along, and also provides structural support. A tissue construct may comprise a matrix contacted with a cell directly after contacting, or may have undergone extensive culturing under conditions suitable for the cell to proliferate and to form an assembly of cells within the matrix that is similar to a tissue found in vivo.

The term “biomatrix,” as used herein, refers to the extracellular matrix of a tissue or organ formed by a living subject. Typically, the extracellular matrix comprises those molecules forming or occupying the space between cells of the tissue or organ, also referred to as the interstitial space, and the basal membranes, also referred to as the basement membranes, if any, of the tissue or organ. The interstitial space typically comprises a three-dimensional network, or mesh, of polysaccharides and fibrous proteins, while the basal membrane are sheet-like depositions of extracellular matrix, which support epithelial cells in living tissues. A biomatrix typically comprises glucosaminoglyans (e.g., proteoglycans, such as heparan sulfate, chondroitin sulfate, and keratan sulfate; and hyaluronic acid), and fibrous proteins (e.g., collagens, such as Collagen types i-XIV), fibronectin, and laminin.

The term “decellularized biomatrix,” as used herein, refers to a tissue from a subject that has been treated to substantially remove living cells, cell membranes, and intracellular components (e.g., cell nuclei and other cell organelles, and cytoplasm). In some embodiments, a decellularized biomatrix is a biomatrix that comprises less than 10%, less than 5%, less than 4%, less than 3%, less than 2%, less than 1%, or less than 0.1% of the number of cells present in the tissue or organ in its native state, e.g., before decellularization. The number of cells present in a tissue or organ before or after decellularization can be assessed by any method known in the art, for example, by staining and counting living cells, or by assessing DNA content as a proxy for cell number. Depending on the assay used, a decellularized biomatrix may be a matrix that comprises less than 10%, less than 5%, less than 4%, less than 3%, less than 2%, less than 1%, or less than 0.1% of the proxy marker, e.g., of DNA, present in the tissue or organ in its native state. Any method for removing cells from a tissue or organ that leave the extracellular matrix of the tissue or organ substantially intact can be used to decellularize a tissue or organ. Typically, a decellularization treatment includes contacting the tissue with a detergent to solubilize the membranes of living cells within the tissue, while leaving the extracellular matrix of the tissue substantially intact. In some embodiments, a vascularized tissue is decellularized by perfusing the tissue or organ with a detergent solution, resulting in a decellularized vascularized biomatrix. Preferably. decellularization of vascularized tissues does not disturb the structural integrity of the basal membrane(s) in vascularized tissues, resulting in a vascularized, decellularized biomatrix with an intact separation of the intravascular space and the interstitial space by the basal membrane. The resulting vascularized, decellularized biomatrix can be perfused similar to the tissue or organ in its native state.

The term “neoplastic cell,” refers to a cell exhibiting an aberrant, hyperproliferative phenotype. Typically, neoplastic cells arise from non-neoplastic tissue cells upon acquiring one or more mutations that support cell division or survival and/or repress tissue homeostasis or cell death. If neoplastic cells arise in vivo, they proliferate in a manner that is excessive as compared to other cells of their tissue of origin. Neoplastic cells can typically be cultured in vitro, and one hallmark feature of many neoplastic cells that can be observed in vitro is their lack of contact inhibition, allowing neoplastic cells to proliferate even once a culture dish has become confluent. This is in contrast to non-neoplastic cells, which do not exhibit a hyperproliferative phenotype.

The term “tumor nodule,” as used herein, refers to an aggregation of tumor cells constituting a lesion, small mass, or lump of tumor cells within a tissue. A tumor nodule typically exhibits a different opacity as compared to surrounding, non-neoplastic tissue, e.g., within the visible light range or the x-ray range, and can be detected with suitable tissue imaging methods. A tumor nodule also typically exhibits a different structural consistency as compared to the surrounding tissue, and, depending on the tissue and the size of the nodule, can be palpable. For example, some tumor nodules can be felt as abnormal lesions within a tissue. In some embodiments, tumor nodules are less than about 3 cm, less than about 2 cm, less than about 1 cm, less than about 5 mm, less than about 2.5 mm, less than about 1 mm, less than about 100 μm, less than about 50 μm, less than about 25 μm, less than about 10 μm, or less than about 5 μm in diameter.

The term “native,” as used herein in the context of the relation of a cell and decellularized biomatrix, refers to a cell that can be found in the tissue or organ that the decellularized biomatrix was derived from. For example, if the decellularized biomatrix is a rat lung biomatrix obtained from a healthy rat lung, then a cell that could be found in a healthy rat lung would be a cell that is native to the decellularized biomatrix. This would include, in this example, rat lung cells, e.g., rat ciliated epithelial cells, rat goblet cells, rat basal cells, and rat brush cells, but not human or mouse lung cells, cells from other tissues (e.g., kidney cells, liver cells, etc.) or neoplastic cells.

The term “healthy,” as used herein in the context of tissues or organs refers to tissues or organs that do not exhibit any signs of disease or disorder, for example, tissues and organs that do not show any signs of tumor formation, neoplasia, dystrophy, or any abnormality as compared to a tissue or organ of the same type obtained or expected to be obtained from a healthy subject, e.g., a subject that has not been diagnosed and/or does not show any sign of a disease or disorder. In some embodiments, a healthy tissue or organ is a tissue or organ obtained from a healthy subject.

The term “biomarker,” as used herein, refers to a measurable parameter, or combination of parameters, that can be used as an indicator of a biological state. The term “biomarker associated with cancer,” accordingly, refers to a biomarker that can be used as an indicator of cancer.

The term “reference value,” as used herein, refers to a pre-determined value to which values obtained from a measurement, for example, a measurement as part of an experimental readout, are compared. In some embodiments, the reference value is a control value, also referred to as a baseline value, which represents the status of an experimental system without the factor to be investigated introduced into the system for an experimental treatment. For example, in an experimental setup in which tumor nodule formation is monitored by contacting a tissue construct comprising neoplastic cells with a candidate drug and assessing the number of tumor nodules formed in the tissue construct at a given experimental endpoint, a suitable control value may be obtained from a tissue construct that is not treated with a candidate drug, or from a plurality of such untreated constructs.

### Introduction

Cancer is one of the leading causes of death in the world and while recent decades have seen much progress in the diagnosis and treatment of various types of cancer, many types of cancer are still associated with a poor survival prognosis once diagnosed. Among cancer deaths in the United States, lung cancer deaths are the most common, with 222,520 patients diagnosed with lung cancer in 2010, and 157,300 patients dying of the disease in the same year [1]. After more than 30 years of research to improve the medical and surgical care of patients with lung cancer, the overall 5-year survival rate for patients with lung cancer has improved only from 13% in 1975 to 16% in 2005 [1]. The situation is similarly dire in other types of cancer.

One major factor in the lack of success in improving patient prognosis and survival after cancer diagnosis is the limitation of current in vitro and in vivo cancer models and, in turn, the limitation of data obtained from studies using the current models, which translate poorly into human clinical practice because of their lack of concordance with the situation present in the human body [2].

Some aspects of this disclosure are based on the recognition that one limitation of current in vitro and in vivo model systems that contributes to this lack of concordance is their shortcoming in modeling the conditions and interactions human neoplastic cells are confronted with in the human body. For example, a Boyden chamber test is commonly used to study the invasive properties of a neoplastic cell population of interest [3]. It measures the ability of cells to migrate across an artificial barrier, which a neoplastic cell will never encounter in a native environment. In the human body, a hallmark of invasiveness is a neoplastic cell's ability to migrate across the basement membrane, which separates the interstitial space from the vascular space of the host organism. Some aspects of this disclosure relate to the recognition that a better model to assess the invasive properties of neoplastic cells would allow an assessment of the ability of cells to migrate across the basement membrane.

Similarly, some aspects of this disclosure are based on the recognition that the highly artificial nature of most in vitro culture systems imposes selective pressures on neoplastic cells that may be irrelevant or even contrary to those encountered by a neoplastic cell or cell population in the human body. Conventional two-dimensional cell culture techniques are highly selective for cells that are able to survive and proliferate in the culture dish. Human tumor cells in vivo, however, do not encounter selective pressure for two-dimensional proliferation and many of the synthetic materials used in in vitro culture, but typically form three-dimensional structures, such as tumor nodules, with drug, metabolite, and cell-cell interaction kinetics much different from those in two-dimensional culture. Three-dimensional in vitro culture models, for example, models using synthetic matrices and matrigel, provide a somewhat more realistic representation of the three-dimensional niches populated by neoplastic cells in vivo than conventional two-dimensional culture dishes, and such models have improved our understanding of some aspects of the interaction of cancer cells with the matrix [4]. However, the use of a synthetic, nonphysiologic matrix, which does not truly mimic native biomatrix conditions, limits the value of data obtained from such culture systems.

In vivo studies, on the other hand, provide data of the interactions of cancer cells with a host organism. However, current in vivo models for the study of human cancer cells typically rely on an immunodeficient host animal, for example, an immunodeficient mouse or rat, and, thus, do not allow neoplastic cells under investigation to interact with human host cells and tissues, but select for cells that can survive and proliferate under the conditions provided by the non-human host. The challenges posed to tumor formation and proliferation in a non-human host may be entirely different from the relevant conditions encountered in the human body. Cancer cells under study in non-human animal models, however, do not encounter human host cell niches and are also not confronted with the human immune response, which limits the value of data obtained from such in vivo models.

Some aspects of this disclosure address and overcome at least some of the shortcomings of the current in vitro and in vivo models of cancer described above. Some aspects of this disclosure are based on the surprising discovery that a decellularized biomatrix obtained from a tissue or organ harvested from a subject can be seeded with neoplastic cells, for example, cancer cells, to obtain a three-dimensional culture of neoplastic cells that more closely resembles a tumor found in vivo than any culture system currently in use, including current 3D culture systems using a synthetic matrix or a matrix shed by cells cultured in vitro on a synthetic matrix scaffold.

Some embodiments described herein provide reagents and methods for the generation of tissue constructs in which neoplastic cells are grown within a decellularized biomatrix. Some embodiments of such tissue constructs more closely resemble tumors in vivo, for example, clinically presenting human tumors, in their structure, their gene expression, their secretion of signaling molecules, and in other aspects. Some embodiments provide tissue constructs in which neoplastic cells are grown within a decellularized biomatrix obtained from a tissue or an organ harvested from a human or a non-human mammal. The tissue constructs described herein allow for the cells grown therein, for example, neoplastic cells to interact with a substrate similar or identical to that present in a subject having a tumor, for example, present in human patients, which, in turn, results in tumor structures, e.g., tumor nodules, that are reminiscent of tumor structures presented in the clinic.

Some aspects of this disclosure are based on the surprising discovery that neoplastic cells can form 3D tumor structures reminiscent of in vivo tumor tissue when cultured within a biomatrix from a different species according to the methods and using the reagents provided herein. For example, human tumor cells have been found to form perfusable tumor nodules when cultured within rat biomatrix, as described in more detail elsewhere herein. Accordingly, the tissue constructs described herein present a unique avenue for investigating cancer biology as well as evaluating clinically relevant parameters of neoplastic cells, for example, of cancer cell lines and of neoplastic cells obtained via a biopsy from a human patient. The tissue constructs described herein also allow for the identification of agents, e.g., chemical compounds, with anti-cancer properties under conditions more realistically resembling in vivo tumor pharmacokinetics and—dynamics than conventional in vitro cancer models.

Some aspects of this disclosure provide methods and reagents for the preparation of decellularized biomatrices from tissues or organs harvested from a subject. Some aspects of this disclosure provide methods and reagents for culturing neoplastic cells within such decellularized biomatrices, for example, human cancer cells. Some aspects of this invention provide methods and reagents for the generation of tissue constructs comprising a decellularized biomatrix seeded with neoplastic cells. Some aspects of this invention provide bioreactors for the generation of tissue constructs. Some aspects of this invention provide methods and reagents for the analysis and evaluation of cancer-associated characteristics in tissue constructs.

The generation of exemplary decellularized biomatrices from mammalian tissue, for example, from rat lung, and the use of these exemplary biomatrices for the generation of lung cancer constructs using a variety of neoplastic cells is described in detail herein. Seeding of decellularized biomatrices with lung cancer cells led to the generation of lung tumor constructs that closely replicated human lung cancer biology, as described in more detail elsewhere herein. The described lung cancer constructs and the methods and reagents for their generation described herein, accordingly, provide a new avenue to better understand lung cancer biology. While this disclosure exemplifies the use of decellularized biomatrices by describing the use of lung biomatrices, it will be understood by those of skill in the art that this exemplification is descriptive and serves merely to illustrate aspects of the described invention, but does not limit the disclosure. It will be apparent to those of skill in the art that decellularized biomatrices can be generated from tissues and organs other than lung and used to generate tumor constructs from neoplastic cells other than the cells and cell lines described herein.

### Tumor Models Using a Decellularized Biomatrix

The term “matrix” refers to the structural component of the cell microenvironment. Matrix is also commonly referred to as “extracellular matrix” or “ECM.” It may be composed of collagens, proteoglycans, laminins, and elastin, which are substances that have been reported to support growth and proliferation of epithelial, mesenchymal, and endothelial cells [5]. Matrix is believed to provide important tumor-stromal interactions and a microenvironment that promotes systematic cell growth in the presence of surrounding growth factors, hormones, and adhesion molecules [6-8].

Recent studies on organ reengineering [9, 10] for orthotopic transplantation have provided a new avenue for isolating naturally occurring biomatrix to use for growing cells in a three-dimensional environment with a preserved extracellular matrix and vasculature system. Analysis of the isolated biomatrix shows that the composition of biomatrices, for example, of the lung matrix, is similar among different species [11]. Moreover, Ott and colleagues [9] have shown that lung cell lines, minced lung tissues, and endothelial cells can grow by means of a combined perfusion- and respiration-based system.

While decellularized biomatrices have been used for the generation of tissues and organs for transplantation, for example, in the context of regenerative medicine, biomatrices have not been used to generate models of diseased tissues, and in particular for the generation of cancer or tumor constructs, and such use has not previously been suggested. It has now surprisingly been found that decellularized biomatrices can be used to generate tissue constructs, for example, lung cancer tissue constructs, that closely resemble in vivo tumor tissues in many aspects, as described in more detail herein.

### Methods for Harvest and Decellularization of Biomatrix

Some aspects of this disclosure provide methods, reagents, and devices for the harvest and decellularization of tissues or organs from mammalian subjects. While the harvest and decellularization of lung biomatrix is described in detail herein to exemplify a method suitable according to aspects of this disclosure, the disclosure is not limited in this respect, and any tissue or organ can be used according to aspects of this invention to generate decellularized biomatrices.

In some embodiments, the tissue or organ is a mammalian tissue or organ. In some embodiments, the tissue or organ is or comprises a lung, pharynx, larynx, trachea, bronchus, diaphragm, heart, blood vessel, esophagus, stomach, liver, gallbladder, pancreas, intestine, colon, rectum, endocrine gland, kidney, ureter, bladder, urethra, lymph node, lymph vessel, tonsil, adenoid, thymus, spleen, skin, muscle, brain, spinal cord, nerve, ovary, uterus, mammary gland, testis, vas deferens, prostate, bone, bone marrow, cartilage, ligament, or tendon tissue or organ.

In some embodiments, a whole organ is harvested and used to generate a decellularized biomatrix. In some embodiments, only part of an organ is harvested and used. In some embodiments, a perfusable organ, organ part, or tissue is harvested and used to generate a decellularized biomatrix.

The tissue, organ, or organ part is harvested from a subject. In some embodiments, the subject is a human. In some embodiments, the subject is a non-human mammal or a non-human vertebrate. In some embodiments, the subject is laboratory animal, a mouse, a rat, a rodent, a farm animal, a pig, a cattle, a horse, a goat, a sheep, a companion animal, a dog a cat, or a guinea pig. Additional suitable subjects for harvesting organs, organ parts, or tissues for use in the methods provided herein will be apparent to those of skill in the art, and the disclosure is not limited in this respect. In some embodiments, the tissue or organ used for generating a decellularized biomatrix is obtained from a cadaver of a subject, for example, from a non-human mammalian cadaver, from a non-human vertebrate cadaver, or from a human cadaver.

In some embodiments, the organ, organ part, or tissue, is harvested from a living subject. In some embodiments, the subject is sacrificed after organ, organ part, or tissue harvest, while in other embodiments, the subject survives the harvest. Survival after harvest from a living subject is feasible, for example, in cases where the organ, organ part, or tissue is not essential for survival of the subject, e.g., in the case of harvesting skin, spleen, thymus, kidney, muscle, bone, bone marrow, cartilage, and other non-essential tissues, organs, or organ parts. Harvest from a living subject typically requires anesthesia of the subject. Methods for anesthesia of subjects, both animals and humans, are well known to those of skill in the medical and scientific arts. Some exemplary anesthesia agents and protocols are described herein, and additional suitable agents and protocols will be apparent to those of skill in the art. This disclosure is not limited in this respect.

In embodiments, where a vascularized tissue, organ, or organ part is harvested for generating a decellularized biomatrix, the harvesting procedure preferably avoids or minimizes blood coagulation or clotting in the vasculature of the harvested tissue, organ, or organ part. This can be achieved, for example, by injecting an anti-coagulant into the blood stream of the subject prior to harvesting the organ, organ part, or tissue. Anti-coagulants suitable for injection are known to those of skill in the art, and exemplary anti-coagulants include, but are not limited to, heparin and heparin derivatives, coumadines (e.g., warfarin, acenocoumarol, phenprocoumon, atromentin, brodifacoum, and phenindione), Factor Xa inhibitors, thrombin inhibitors, batroxobin, hementin, and Ca²⁺-ion chelators (e.g., EDTA, citrate, oxalate). In some embodiments, the organ, organ part, or tissue to be harvested is perfused with a fluid replacing any blood in the vasculature of the organ, organ part, or tissue. Typically, the perfusion fluid comprises a buffering agent (e.g., PBS), and an anticoagulant.

Surgical methods for the isolation of an organ, organ part, or tissue that are suitable for use according to aspects of this invention are well known in the art. Some exemplary methods are described herein, and additional suitable methods will be apparent to the skilled artisan. Suitable methods include, but are not limited to, surgical methods used for the harvest of organs, organ parts, or tissues for transplantation or for use in tissue engineering.

Methods for perfusion of a vascularized organ, organ part, or tissue, prior to, during, and after harvest are well known in the art. Such methods typically include guiding a flow of perfusion fluid through the vasculature of the organ, organ part, or tissue. This can be achieved by connecting a perfusion influx to a blood vessel of the organ, organ part, or tissue, and providing a vent for the perfusion fluid to exit the organ, organ part, or tissue. In some embodiments, the perfusion influx is connected to an artery of the organ, organ part, or tissue. In some embodiments, the vent comprises a puncture of an effluent vessel, for example, a vein, of the organ, organ part, or tissue. In some embodiments, a perfusion efflux is connected to an effluent vessel of the organ, organ part, or tissue. Providing both a perfusion influx and a perfusion efflux allows close control of perfusion fluid flow rate and pressure within the organ, organ part, or tissue. In some embodiments, perfusion influx and/or efflux are provided in the form of a cannula, tube, or a standard connector format, such as a Luer bulkhead. Perfusion influx and/or efflux are typically connected to a container comprising perfusion fluid, for example, via flexible tubing, such as Tygon tubing. Perfusion systems suitable according to aspects of this disclosure typically also comprise a pump, for example, a peristaltic pump, moving the perfusion fluid through the influx and/or the efflux at a certain flow rate.

Harvest of the organ, organ part, or tissue typically includes removal of the organ, organ part, or tissue, from the subject, and transfer to a sterile or semi-sterile environment. After the organ, organ part, or tissue is harvested, and, where suitable, the vasculature of the organ, organ part, or tissue is connected to a perfusion system, the decellularization process can be initiated.

Some embodiments provide methods, reagents, and devices for decellularizing an organ, organ part, or tissue. In some embodiments, decellularization comprises contacting an isolated organ, organ part, or tissue with an agent that removes cells, but leaves the ECM of the organ, organ part, or tissue intact, thus resulting in a decellularized ECM. In some embodiments, a decellularized biomatrix is free of cells. In other embodiments, a decellularized biomatrix comprises less than 5%, less than 4%, less than 3%, less than 2%, less than 1%, or less than 0.1% of the cells comprised in the harvested organ, organ part, or tissue at the time of harvest.

In some embodiments, a method of decellularization is provided that includes contacting the organ, organ part, or tissue with a detergent at a concentration and for a time effective to solubilize cell membranes and/or other intracellular components, such as cell organelles, cell nuclei, and cellular DNAs and RNAs. Suitable detergents for cell solubilization are well known to those of skill in the art and non-limiting examples of suitable detergents include sodium dodecyl sulfate (SDS), Tween 20, Triton X-100, Triton X-101, NP40, and similar detergents. In some embodiments, the method of decellularization comprises perfusing the harvested organ, organ part, or tissue with a wash solution, for example, with a buffer comprising an anti-coagulant. In some embodiments, the method comprises contacting (e.g., perfusing) the organ, organ part, or tissue with a detergent solution, e.g., with a solution comprising SDS, Tween 20, Triton X-100, Triton X-101, and/or NP40, at a total detergent concentration within the range of about 0.01%—about 10%, for example, of about 0.01%, about 0.02%, about 0.05%, about 0.1%, about 0.2%, about 0.3%, about 0.4%, about 0.5%, about 1%, about 2%, about 5%, or about 10%. In some embodiments, the harvested organ, organ part, or tissue is contacted (e.g., perfused) with the detergent solution for at least 30 min, at least 1 hour, at least 2 hours, at least 4 hours, at least 8 hours, at least 12 hours, overnight, or at least 24 hours. In some embodiments, the decellularization process is monitored, for example, by assaying the effluent detergent solution used for contacting the organ, organ part, or tissue for a cellular component (e.g., cellular DNA). In some embodiments, the decellularization process is stopped based on quantifying the cellular component in the detergent solution used for contacting the organ, organ part, or tissue, for example, the decellularization process may be stopped once the level of the cellular component in the effluent detergent solution falls below a threshold value (e.g., equal to or less than a reference value, less than the detection limit, or less than 5%, less than 1%, less than 0.1% of a reference value obtained from a measurement taken immediately after the initial contacting of the organ, organ part, or tissue with the detergent solution). In some embodiments, a plurality of different detergents is used for decellularization, either in combination or sequentially. In some embodiments, residual detergent left after decellularization is removed by washing the decellularized biomatrix with a wash solution, for example, with water, or with a perfusion fluid.

In some embodiments, a decellularized biomatrix is sterilized by contacting (e.g., perfusing) it with a solution comprising one or more antibiotic agents at a concentration and for a time effective to kill any remaining cells or biological contaminants present on the decellularized biomatrix. Suitable antibiotics, concentrations, and time periods are well known to those of skill in the art. Decellularized biomatrices, whether sterilized or not, can be used immediately for seeding with cells, e.g., with human neoplastic cells, or can be stored for later use. In some embodiments, decellularized biomatrices are stored at room temperature, at 4° C., on blue ice, at 0° C., at −20° C., at −80° C., on dry ice, or in liquid nitrogen.

Decellularized biomatrices prepared according to method herein from vascularized organs, organ parts, or tissues, typically retain the integrity and/or the separation of the vascular space, also referred to herein as the intravascular space, comprising the space including the vascular lumen and circumscribed by the walls, or basal membranes, of the blood vessels, and the extravascular space, sometimes also referred to as the tissue space, and comprising the interstitial space and any space occupied by cells in the original organ, organ part or tissue. In some embodiments, the separation of intravascular and extravascular space allows continued perfusion of the decellularized biomatrix without significant leakage into the extravascular space, and/or contacting of the extravascular space with cells to be seeded onto the decellularized biomatrix.

### Customized Decellularization Chambers

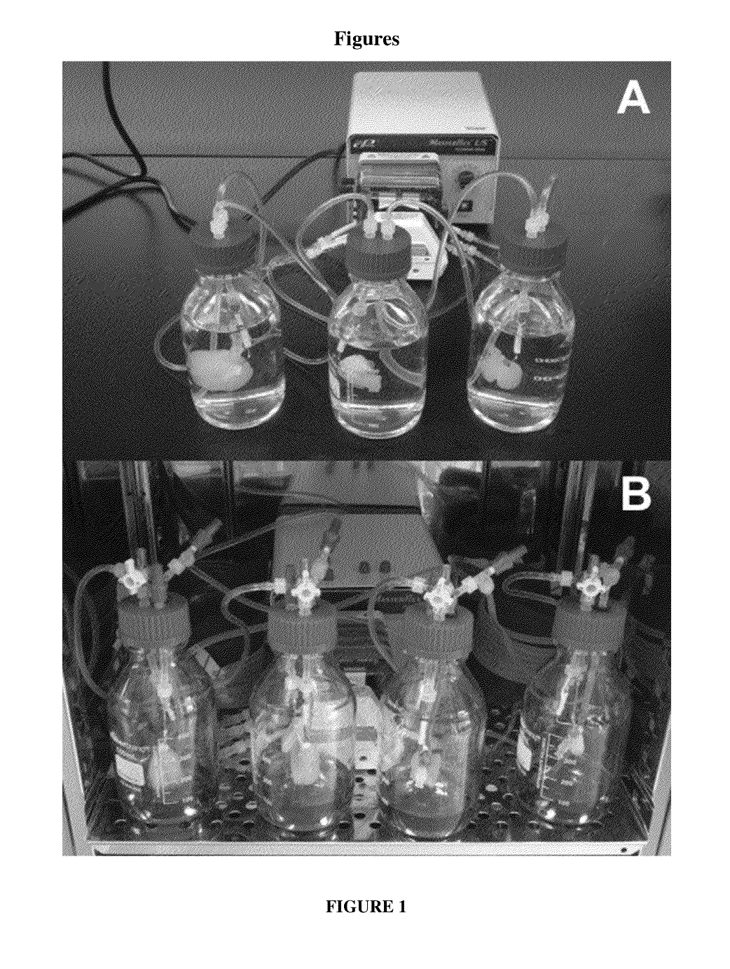

Some embodiments provide a decellularization chamber, as exemplified in FIG. 1A, which depicts a chamber useful for decellularization of small vascularized organs, organ parts, or tissues, for example, harvested from laboratory animals. The exemplary chamber can be scaled up or down to accommodate larger or smaller organs, organ parts, or tissues. In some embodiments, a decellularization chamber is provided that comprises a container, for example a 500-mL glass bottle, having an influx which can be connected to an artery of the organ, organ part, or tissue to be decellularized, and an efflux which can be used to remove any liquid from the container. For decellularization, the organ, organ part, or tissue is placed into the container, and the influx is connected to an artery of the organ, organ part, or tissue. Fluids useful for decellularization, e.g., detergent solutions and wash solutions, are then introduced into the organ, organ part, or tissue via the influx until decellularization is achieved, and any fluid flowing out of the organ, organ part, or tissue is removed from the container via the efflux. In some embodiments, influx and/or efflux comprise standard adapters, for example Luer adapters, which facilitates the connection of different liquid reservoirs holding the solutions used for decellularization. In some embodiments, the decellularization chamber comprises a pump for introducing the various decellularization liquids into the organ, organ part, or tissue. In some embodiments, the decellularization chamber further comprises a pressure gauge allowing for the fluids to be introduced and maintained within the organ, organ part, or tissue at a certain pressure.

### Tissue Constructs

Some embodiments provide tissue constructs comprising decellularized biomatrix. In some embodiments, the tissue construct comprises a decellularized biomatrix as provided herein, for example, a decellularized biomatrix obtained according to methods provided herein, and a population of neoplastic cells seeded onto the decellularized biomatrix.

In some embodiments, the tissue construct comprises neoplastic cells that adhere to, populate, and/or proliferate on the decellularized biomatrix. In some embodiments, the tissue construct comprises neoplastic cells that have been cultured on the decellularized biomatrix for at least 1 h, at least 2 h, at least 4 h, at least 8 h, at least overnight, at least 12 h, at least 24 h, at least 2 d, at least 3 d, at least 4 d, at least 5 d, at least 6 d, at least 7 d, at least one week, at least two weeks, at least 3 weeks, at least 4 weeks, at least 1 month, at least 2 months, or longer than 2 months.

A neoplastic cell population is a cell population that exhibits aberrant, or abnormal, proliferation characteristics as compared to a normal cell population. Such aberrant proliferation characteristics may include, but are not limited to, lack of contact inhibition, increased life span, increased cell division rate, increased cell survival when faced with apoptotic stimuli, and sustained proliferation in the absence of external proliferation stimuli. Neoplastic cell populations include, but are not limited to tumor cell populations, cancer cell populations, benign neoplastic cell populations, and malign cell populations. Neoplastic cell populations include cell populations obtained from human tumors or cancers, for example, via biopsy. Examples of neoplastic cell populations are cell populations, including primary cell populations and cell lines, obtained from cancer, including invasive and non-invasive cancer, for example, lung cancer (e.g., small-cell lung cancer, non-small cell lung cancer), skin cancer (e.g., melanoma), stomach cancer, liver cancer, colorectal cancer, breast cancer, pancreatic cancer, prostate cancer, blood cancer, bone cancer, bone marrow cancer, and other cancers.

Suitable neoplastic cell lines for the generation of tissue constructs according to aspects of this invention are well known to those of skill in the art. Such cell lines include, but are not limited to the following cell lines (Official Nomenclature according to the National Center for Biotechnology Information and the Wellcome Trust Sanger Institute): A101D, A172, A204, A2058, A253, A2780, A3-KAW, A375, A388, A4-Fuk, A427, A431, A498, A549, A673, A704, ABC-1, ACHN, ACN, AGS, ALL-PO, AM-38, AML-193, AN3-CA, ARH-77, ATN-1, AU565, AsPC-1, BALL-1, BB30-HNC, BB49-HNC, BB65-RCC, BC-1, BC-3, BCPAP, BE-13, BEN, BFTC-905, BFTC-909, BHT-101, BHY, BL-41, BL-70, BOKU, BONNA-12, BPH-1, BT-20, BT-474, BT-549, BV-173, Becker, BxPC-3, C-33-A, C-4-11, C2BBe1, C32, C3A, C8166, CA46, CADO-ES1, CAKI-1, CAL-120, CAL-12T, CAL-148, CAL-27, CAL-33, CAL-39, CAL-51, CAL-54, CAL-62, CAL-72, CAL-85-1, CAMA-1, CAPAN-1, CAS-1, CCF-STTG1, CCRF-CEM, CESS, CFPAC-1, CGTH-W-1, CHL-1, CHP-126, CHP-134, CHP-212, CMK, CML-T1, COLO-205, COLO-320-HSR, COLO-668, COLO-678, COLO-679, COLO-680N, COLO-684, COLO-741, COLO-792, COLO-800, COLO-824, COLO-829, COR-L105, COR-L23, COR-L279, COR-L88, COR-L96CAR, CP50-MEL-B, CP66-MEL, CP67-MEL, CPC-N, CRO-AP2, CRO-APS, CTB-1, CTV-1, CW-2, Ca-Ski, Ca9-22, CaR-1, Calu-1, Calu-3, Calu-6, Caov-3, Caov-4, Capan-2, ChaGo-K-1, CoCM-1, D-245MG, D-247MG, D-263MG, D-283MED, D-336MG, D-384MED, D-392MG, D-397MG, D-423MG, D-458MED, D-502MG, D-538MG, D-542MG, D-556MED, D-566MG, DB, DBTRG-05MG, DEL, DG-75, DJM-1, DK-MG, DMS-114, DMS-153, DMS-273, DMS-53, DMS-79, DOHH-2, DOK, DSH1, DU-145, DU-4475, DV-90, Daoy, Daudi, Detroit562, DoTc2-4510, EB-3, EB2, EC-GI-10, ECC10, ECC12, ECC4, EFE-184, EFM-19, EFO-21, EFO-27, EGI-1, EHEB, EKVX, EM-2, EPLC-272H, ES1, ES3, ES4, ES5, ES6, ES7, ES8, ESS-1, ETK-1, EVSA-T, EW-1, EW-11, EW-12, EW-13, EW-16, EW-18, EW-22, EW-24, EW-3, EW-7, EoL-1-cell, FADU, FTC-133, G-361, G-401, G-402, GA-10-Clone-4, GAK, GAMG, GB-1, GCIY, GCT, GDM-1, GI-1, GI-ME-N, GMS-10, GOTO, GP5d, GR-ST, GT3TKB, H-EMC-SS, H4, H9, HA7-RCC, HAL-01, HC-1, HCC1143, HCC1187, HCC1395, HCC1419, HCC1569, HCC1599, HCC1806, HCC1937, HCC1954, HCC2157, HCC2218, HCC2998, HCC38, HCC70, HCE-4, HCE-T, HCT-116, HCT-15, HD-MY-Z, HDLM-2, HEC-1, HEL, HGC-27, HH, HL-60, HLE, HMV-II, HN, HO-1-N-1, HOP-62, HOP-92, HOS, HPAF-II, HSC-2, HSC-3, HSC-4, HT, HT-1080, HT-1197, HT-1376, HT-144, HT-29, HT-3, HT55, HTC-C3, HUH-6-clone5, HUTU-80, HeLaSF, Hs-578-T, HuCCT1, HuH-7, HuO-3N1, HuO9, HuP-T3, HuP-T4, IA-LM, IGR-1, IGROV-1, IM-9, IMR-5, IPC-298, IST-MEL1, IST-MES1, IST-SL1, IST-SL2, ITO-II, J-RT3-T3-5, J82, JAR, JEG-3, JVM-2, JVM-3, JiyoyeP-2003, K-562, K052, K5, KALS-1, KARPAS-299, KARPAS-422, KARPAS-45, KASUMI-1, KATO111, KE-37, KG-1, KGN, KINGS-1, KLE, KM-H2, KM12, KMOE-2, KMS-12-PE, KNS-42, KNS-62, KNS-8′-FD, KOSC-2, KP-4, KP-N-RT-BM-1, KP-N-S19s, KP-N-YN, KP-N-YS, KS-1, KU-19-19, KU812, KURAMOCHI, KY821, KYSE-140, KYSE-150, KYSE-180, KYSE-270, KYSE-410, KYSE-450, KYSE-510, KYSE-520, KYSE-70, L-363, L-428, L-540, LAMA-84, LAN-6, LB1047-RCC, LB2241-RCC, LB2518-MEL, LB373-MEL-D, LB647-SCLC, LB771-HNC, LB831-BLC, LB996-RCC, LC-1F, LC-2-ad, LC4-1, LCLC-103H, LCLC-97TM1, LK-2, LN-405, LNCaP-Clone-FGC, LOUCY, LOXIMVI, LP-1, LS-1034, LS-123, LS-174T, LS-411N, LS-513, LU-134-A, LU-135, LU-139, LU-165, LU-65, LU-99A, LXF-289, LoVo, M059J, M14, MC-1010, MC-CAR, MC-IXC, MC116, MCF7, MDA-MB-134-VI, MDA-MB-157, MDA-MB-175-VII, MDA-MB-231, MDA-MB-361, MDA-MB-415, MDA-MB-435, MDA-MB-453, MDA-MB-468, ME-180, MEG-01, MEL-HO, MEL-JUSO, MES-SA, MFE-280, MFE-296, MFH-ino, MFM-223, MG-63, MHH-CALL-2, MHH-CALL-4, MHH-ES-1, MHH-NB-11, MHH-PREB-1, MIA-PaCa-2, MJ, MKN1, MKN28, MKN45, MKN7, ML-2, MLMA, MMAC-SF, MN-60, MOLT-13, MOLT-16, MOLT-4, MONO-MAC-6, MPP-89, MRK-nu-1, MS-1, MSTO-211H, MUTZ-1, MV-4-11, MZ1-PC, MZ2-MEL, MZ7-mel, Malme-3M, Mewo, Mo-T, NALM-1, NALM-6, NB1, NB10, NB12, NB13, NB14, NB17, NBS, NB6, NB69, NB7, NBsusSR, NCCIT, NCI-ADR-RES, NCI-H1048, NCI-H1092, NCI-H1105, NCI-H1155, NCI-H1173, NCI-H1184, NCI-H128, NCI-H1284, NCI-H1299, NCI-H1304, NCI-H1355, NCI-H1395, NCI-H1417, NCI-H1436, NCI-H1437, NCI-H146, NCI-H1522, NCI-H1563, NCI-H157, NCI-H1573, NCI-H1581, NCI-H1618, NCI-H1623, NCI-H1648, NCI-H1650, NCI-H1651, NCI-H1666, NCI-H1693, NCI-H1694, NCI-H1703, NCI-H1734, NCI-H1755, NCI-H1770, NCI-H1792, NCI-H1793, NCI-H1838, NCI-H187, NCI-H1882, NCI-H1926, NCI-H1930, NCI-H1963, NCI-H1975, NCI-H1993, NCI-H2009, NCI-H2029, NCI-H2030, NCI-H2052, NCI-H2081, NCI-H2087, NCI-H209, NCI-H2107, NCI-H2122, NCI-H2126, NCI-H2141, NCI-H2170, NCI-H2171, NCI-H2196, NCI-H2227, NCI-H2228, NCI-H226, NCI-H2291, NCI-H23, NCI-H2330, NCI-H2342, NCI-H2347, NCI-H2405, NCI-H2452, NCI-H250, NCI-H28, NCI-H292, NCI-H295, NCI-H322M, NCI-H345, NCI-H358, NCI-H378, NCI-H441, NCI-H446, NCI-H460, NCI-H508, NCI-H510A, NCI-H520, NCI-H522, NCI-H524, NCI-H526, NCI-H596, NCI-H630, NCI-H64, NCI-H650, NCI-H661, NCI-H69, NCI-H711, NCI-H716, NCI-H719, NCI-H720, NCI-H727, NCI-H747, NCI-H748, NCI-H774, NCI-H810, NCI-H82, NCI-H835, NCI-H838, NCI-H889, NCI-N417, NCI-N87, NCI-SNU-1, NCI-SNU-16, NCI-SNU-5, NEC8, NH-12, NH-6, NKM-1, NMC-G1, NOMO-1, NOS-1, NTERA-S-cl-D1, NUGC-3, NY, no-10, no-11, OAW-28, OAW-42, OC-314, OCI-AML2, OCUB-M, OE19, OE33, OMC-1, ONS-76, OPM-2, OS-RC-2, OVCAR-3, OVCAR-4, OVCAR-5, OVCAR-8, P12-ICHIKAWA, P30-OHK, P31-FUJ, PA-1, PANC-03-27, PANC-08-13, PANC-10-05, PC-14, PC-3, PF-382, PFSK-1, PLC-PRF-5, PSN1, QIMR-WIL, RCC10RGB, RCM-1, RD, REH, RERF-LC-FM, RERF-LC-MS, RF-48, RH-1, RH-18, RKO, RL, RL95-2, RMG-I, R082-W-1, RPMI-2650, RPMI-6666, RPMI-7951, RPMI-8226, RPMI-8402, RPMI-8866, RS4-11, RT-112, RT4, RTSG, RVH-421, RXF393, Raji, Ramos-2G6-4C10, S-117, SAS, SBC-1, SBC-5, SCC-15, SCC-25, SCC-3, SCC-4, SCC-9, SCCH-26, SCH, SCLC-21H, SF126, SF268, SF295, SF539, SH-4, SHP-77, SIG-M5, SIMA, SJRH30, SJSA-1, SK-CO-1, SK-HEP-1, SK-LMS-1, SK-LU-1, SK-MEL-1, SK-MEL-2, SK-MEL-24, SK-MEL-28, SK-MEL-3, SK-MEL-30, SK-MEL-5, SK-MES-1, SK-MG-1, SK-MM-2, SK-N-AS, SK-N-DZ, SK-N-FI, SK-NEP-1, SK-OV-3, SK-PN-DW, SK-UT-1, SKG-IIIa, SKM-1, SN12C, SNB19, SNB75, SNG-M, SNU-387, SNU-423, SNU-449, SNU-475, SNU-C1, SNU-C2B, SR, ST486, SU-DHL-1, SUP-B8, SUP-T1, SW1088, SW1116, SW13, SW1417, SW1463, SW1573, SW1710, SW1783, SW1990, SW48, SW620, SW626, SW684, SW756, SW780, SW837, SW872, SW900, SW948, SW954, SW962, SW982, Saos-2, SiHa, T-24, T47D, T84, T98G, TALL-1, TC-YIK, TCCSUP, TE-1, TE-10, TE-11, TE-12, TE-15, TE-161-T, TE-206-T, TE-441-T, TE-5, TE-6, TE-8, TE-9, TGBC11TKB, TGBC1TKB, TGBC24TKB, TGW, THP-1, TI-73, TK10, TT, TUR, TYK-nu, U-118-MG, U-2-OS, U-266, U-698-M, U-87-MG, UO31, U251, UACC-257, UACC-62, UACC-812, UACC-893, UM-UC-3, UMC-11, VA-ES-BJ, VM-CUB-1, VMRC-MELG, VMRC-RCZ, WERI-Rb-1, WM-115, WSU-NHL, YAPC, YH-13, YKG-1, YT, ZR-75-30, 22RV1, 23132-87, 380, 5637, 639-V, 647-V, 697, 769-P, 786-0, 8-MG-BA, 8305C, 8505C. Suitable cell lines for the generation of tissue constructs according to aspects of this invention further comprise any neoplastic cell line, tumor cell line, or cancer cell line described in Romano, Maniello, Aresu, et al., Cell Line Data Base: structure and recent improvements towards molecular authentication of human cell lines, Nucl. Acids Res. (2009) 37 (suppl 1): D925-D932; doi: 10.1093/nar/gkn730; published online Oct. 15, 2008; the entire contents of which are incorporated herein by reference.

In some embodiments, the decellularized biomatrix employed in the generation of the tissue construct is matched to the organ of origin of the neoplastic cell population seeded onto the biomatrix. For example, in some embodiments, a lung cancer cell line is seeded onto a lung decellularized biomatrix, e.g, a decellularized biomatrix obtained from a lung, a lung part (e.g., a pulmonary lobe, or part of a pulmonary lobe), or lung tissue. In other embodiments, a liver cancer cell line is seeded onto a liver decellularized biomatrix. In still other embodiments, a kidney cancer cell is seeded onto a kidney decellularized biomatrix, and so forth. Without wishing to be bound by theory, it is believed that such biomatrix-cell line-matched tissue constructs most closely resemble the natural state of the respective tumor, since the neoplastic cells interact with a biomatrix resembling the matrix of the organ of origin of the cells.

In some embodiments, the decellularized biomatrix employed in the generation of the tissue construct is not matched to the organ of origin of the neoplastic cell population seeded onto the biomatrix. For example, in some embodiments, a skin cancer cell line is seeded onto a lung decellularized biomatrix. In other embodiments, a liver cancer cell line is seeded onto a lung decellularized biomatrix. In still other embodiments, a lung cancer cell is seeded onto a liver decellularized biomatrix, and so forth. The generation of such unmatched tissue constructs allows to investigate organ-specific cell-matrix interactions. Unmatched tissue constructs are also useful to study metastatic cancer biology. For example, unmatched tissue constructs can be used to investigate interactions of metastatic cells with biomatrix of an unmatched tissue.

In some embodiments, the decellularized biomatrix employed in the generation of the tissue construct is matched to the species of origin of the neoplastic cell population seeded onto the biomatrix. For example, in some embodiments, a human cancer cell line is seeded onto a human decellularized biomatrix. In other embodiments, a mouse cancer cell line is seeded onto a mouse decellularized biomatrix. In still other embodiments, a rat cancer cell is seeded onto a rat decellularized biomatrix, and so forth.

In some embodiments, the decellularized biomatrix employed in the generation of the tissue construct is not matched to the species of origin of the neoplastic cell population seeded onto the biomatrix. For example, in some embodiments, a human cancer cell line is seeded onto a rat or mouse decellularized biomatrix. In other embodiments, a mouse cancer cell line is seeded onto a rat decellularized biomatrix. In still other embodiments, a human cancer cell is seeded onto a pig, goat, sheep, dog, cat, or non-human primate decellularized biomatrix, and so forth.

In some embodiments, a tissue construct is provided that is perfusable. Typically, a perfusable tissue construct according to aspects of this disclosure comprises a decellularized biomatrix derived from a vascularized tissue, organ, or organ part. In some embodiments, the vascularized tissue, organ, or organ part comprises at least one artery and/or at least one vein suitable for connecting a perfusion influx and/or a perfusion efflux, respectively. In some embodiments, a tissue construct is provided that is perfused. Such constructs typically comprise a decellularized biomatrix derived from a vascularized tissue, organ, or organ part, which comprises at least one artery and/or at least one vein connected to a perfusion influx and/or a perfusion efflux, respectively. In some embodiments, the perfused construct comprises perfusion fluid at a certain pressure, for example, at physiological pressure.

In some embodiments, a tissue construct is provided that comprises at least one tumor nodule. A tumor nodule may be a small node, for example, a lesion with a diameter in the range of 1-30 mm. In some embodiments, tumor nodules are characterized by a distinct tissue opacity to visible light or as viewed by radiography or other imaging methods, as compared to the surrounding, non-tumorigenic tissue. In some embodiments, tumor nodules are palpable lesions. In some embodiments, a tissue construct is provided that comprises a perfusable tumor nodule. A perfusable tumor nodule, in some embodiments, comprises a tumor nodule that is vascularized, and, thus, accessible to agents introduced into the tissue construct via a perfusion fluid. Such perfusable tumor nodules are useful for investigating tumor biology as well as pharmacokinetics and—dynamics of specific tumor nodules, and for identifying anti-cancer agents, for example, using methods of compound screening as described elsewhere herein. Without wishing to be bound by any theory, it is believed that such perfusable tumor nodules resemble the natural state of tumors found in vascularized tissues more closely than non-perfusable nodules, or conventional two-dimensional or three-dimensional cancer models, since human tumors presented in the clinic typically comprise perfusable nodules.

In some embodiments, a tissue construct is provided that comprises a neoplastic cell population and an additional, non-neoplastic cell population seeded onto a decellularized biomatrix. The neoplastic and the non-neoplastic cell populations can be seeded onto the biomatrix together, for example, as a mixture, or sequentially, for example, the non-neoplastic cell population can be seeded first and then the neoplastic cell population, or vice versa. In some embodiments, both cell populations are seeded into the tissue space of the decellularized biomatrix, whereas, in other embodiments, the neoplastic cells are seeded into the tissue space and the non-neoplastic cells are seeded into the intravascular space. In some of the latter embodiments, the non-neoplastic cells are endothelial cells, or are of a cell type that can populate the blood vessel side of the basal membrane separating the tissue and the intravascular space of the decellularized biomatrix.

In some embodiments, the non-neoplastic cell population comprises cells that are origin-matched to the decellularized biomatrix, for example, non-neoplastic lung tissue cells, lung stromal cells, or lung epithelial cells, are seeded onto a decellularized lung biomatrix, non-neoplastic liver cells, hepatocytes, or fibroblasts are seeded onto a liver biomatrix, and so on. Those of skill in the art will understand that these examples are non-limiting, and suitable, additional non-neoplastic cells and cell populations of the mentioned tissues and of other tissues as well as methods for obtaining such cell populations, including, for example, tissue homogenization or lavage, will be apparent to the skilled artisan. Non-neoplastic cell populations may allow neoplastic cells to form cell-cell interactions similar to those formed in the human patient, which is believed to further improve the level of similarity of the tissue constructs described herein to clinically present human tumors.

In some embodiments, the neoplastic cell population comprises metastatic cells, or cells capable of metastasizing, or the neoplastic cell population is derived from a tumor, cancer, or cell line known or suspected to be able to metastasize. In some embodiments, such cells are seeded into the tissue space of the decellularized biomatrix. In some such embodiments, the ability of the cells to transgress the basal membrane separating the tissue and vascular space of the decellularized biomatrix is assessed as a biomarker for the metastasizing capabilities of the cells. It is believed that the basal membrane of a decellularized biomatrix as described herein represents a more realistic barrier to metastasis than the synthetic barriers in conventional assays measuring metastasizing capabilities of cells, which, in turn, provides more relevant data for evaluating the metastasizing potential or capability of cell populations obtained in the course of cancer diagnosis or treatment, such as via clinical biopsies.

In some embodiments, a tissue construct is provided that can be used as a metastatic model. In some such embodiments, the tissue construct comprises a first interstitial space that is seeded with neoplastic cells, also referred to herein as the primary interstitial space, and one or more additional interstitial space(s) not seeded with neoplastic cells, also referred to herein as secondary interstitial space(s). In some embodiments, the primary and secondary interstitial spaces are separate from each other, but share the same vascular space, e.g., in that the basement membranes circumscribing each interstitial space abut the same vasculature or the same vascular space. In some embodiments, interstitial spaces sharing the same vascular space are vascularized interstitial spaces that are perfused, through their vasculature, by the same perfusion medium. In some embodiments, the only way for a neoplastic cell to reach the second interstitial space is via transgression of the basement membrane separating the first interstitial space from the vascular space, thus becoming a circulating cell, and by subsequent transgression of the basement membrane separating the second interstitial space from the vascular space.

In some embodiments a metastatic model biomatrix provided herein is derived from a single organ (e.g., a single lung, kidney, liver, brain, stomach, intestine, pancreas, skin, or bladder) by creating two separate interstitial spaces within the biomatrix. For example, in some embodiments, a decellularized lung biomatrix is provided in which one of the two bronchi of a single lung is seeded with neoplastic cells via the trachea, thus creating a first interstitial space which is seeded with neoplastic cells (a primary interstitial space), while the other bronchus does not receive cells via the trachea, e.g., by virtue of being tied off (ligated), or otherwise separated from tracheal influx, prior to tracheal seeding of cells, thus creating a second interstitial space which is not seeded with neoplastic cells (a secondary interstitial space). In some embodiments, the second interstitial space in a lung biomatrix may be created by separating a lobe, instead of a bronchus, from tracheal influx. The interstitial space of other organs can similarly be divided into two (or more) separate interstitial spaces by separating a sub-structure of the organ, e.g., a liver lobe, a kidney lobe, or a brain hemisphere. The separation can be by tying off (ligating) a sub-part of an organ, or a biomatrix derived from an organ, e.g., a lobe or a hemisphere, or by otherwise physically separating sub-parts of an organ. Additional suitable methods for physical separation of organ sub-parts that can be used to create separate interstitial spaces within a biomatrix are known to those of skill in the art, and the invention is not limited in this respect.

In some embodiments a metastatic model biomatrix provided herein is derived from two or more organs. In some embodiments, one organ, referred to herein as the primary organ, is seeded with neoplastic cells, while the other organ(s), referred to herein as the secondary organ(s), is/are not. In some embodiments, the primary and secondary organs are perfused by flowing the same medium through the vascular space of the organs, thus allowing any circulating cells originating from the primary organ to access the basement membrane of the secondary organ(s). In some embodiments, the primary and secondary organs are of the same organ type. For example, in some embodiments, the primary organ is a lung, and the secondary organ is also a lung. Such metastatic models can be used, for example, to study metastasis within the same organ type, e.g., metastasis from a primary lung tumor into the lung. In other embodiments, a metastatic tumor model is provided in which a secondary organ is of a different type than the primary organ. For example, in some embodiments, the primary organ is a lung, while the secondary organ is a kidney, liver, brain, stomach, intestine, pancreas, skin, bone marrow, or bladder. In other embodiments, the primary organ is a liver, while the secondary organ is a lung, kidney, liver, brain, stomach, intestine, pancreas, skin, bone marrow, or bladder. Other organ combinations are also envisioned, but not listed here for the purpose of brevity. Metastatic models comprising two or more different organs can be used, for example, to study metastatic processes in which metastatic cells target an organ different from the organ of origin, and also to study organ preferences of circulating cells.

### Methods for the Generation of Tissue Constructs

Some embodiments provide methods and reagents for the generation of tissue constructs. In some embodiments, the method comprises providing a decellularized biomatrix, and contacting the decellularized biomatrix with a population of neoplastic cells. In some embodiments, the neoplastic cells are tumor cells or cancer cells, cells from a cancer cell line, cells obtained from a cancer or tumor biopsy, or mutant cells predisposed to form tumors or tumor nodules, e.g., cells overexpressing an oncogene, or deficient for a tumor suppressor. In embodiments, the neoplastic cells are seeded and incubated after seeding under conditions suitable for the neoplastic cells to populate the decellularized biomatrix. Exemplary conditions and time periods are provided herein, and additional suitable conditions and time periods will be apparent to the skilled artisan. The disclosure is not limited in this respect.

In some embodiments, the method comprises obtaining a decellularized biomatrix. In some embodiments, obtaining comprises decellularizing a tissue, organ, or organ part that was obtained from a subject. In some embodiments, obtaining comprises harvesting an organ, organ part, or tissue from a subject and decellularizing the organ, organ part, or tissue to yield a decellularized biomatrix. In some embodiments, obtaining comprises retrieving a previously prepared decellularized biomatrix from a storage or shipping container. In some embodiments, where a previously prepared and/or stored decellularized biomatrix is used, the method may comprise transferring the biomatrix from storage conditions to conditions suitable for perfusing the biomatrix and/or seeding the biomatrix with cells. This may include thawing the biomatrix, washing the biomatrix, soaking the biomatrix, perfusing the biomatrix, and/or otherwise equilibrating the biomatrix in conditions suitable for cell seeding. In some embodiments, obtaining a decellularized biomatrix may include any of the steps, acts, or methods described in the context of preparing a decellularized biomatrix herein.

In some embodiments, contacting the biomatrix with neoplastic cells comprises contacting the tissue space of the biomatrix with a suspension of the cells. In some embodiments, the cell suspension is a single-cell suspension. In some embodiments, the suspension is prepared by providing a culture of the neoplastic cells, enzymatically, and/or mechanically separating individual cells contained in the culture, and transferring the individualized cells into a medium suitable for contacting the decellularized biomatrix at a concentration suitable for cell seeding. Exemplary media for contacting the decellularized biomatrix include cell culture media and buffers. Some exemplary media and cell concentrations suitable for contacting the decellularized biomatrix are provided herein, and additional suitable media and concentrations will be apparent to those of skill in the art.

In some embodiments, the method comprises perfusing the decellularized biomatrix. Typically, the perfused space is the vascular space of the biomatrix, and the perfusion fluid comprises nutrients supporting the population of the biomatrix with the neoplastic cells. In some embodiments, the decellularized biomatrix is perfused and contacted with the neoplastic cells in a bioreactor as described herein. Preferably, the contacting and/or perfusion of the biomatrix is carried out under sterile conditions or semi-sterile conditions. Supplementation of the perfusion fluid or the media comprising the cells with an antibiotic may minimize the chance of biological contamination of the biomatrix or any of the fluids used.

In some embodiments, the decellularized biomatrix used in the method is a lung, a kidney, a liver, a brain, a stomach, an intestine, a pancreas, a skin, a bladder, a bone marrow, or a mucosal biomatrix. In some embodiments, the decellularized biomatrix is a human, mouse, rat, sheep, goat, pig, dog, cat, non-human primate, mammalian, non-human mammalian, or non-human vertebrate biomatrix. In some embodiments, the neoplastic cells used for seeding the biomatrix are malignant cells, cancer cells, tumor cells, cells derived from a primary tumor, cells derived from a secondary tumor or a metastasis, primary cells obtained from a tumor or cancer biopsy, or cells derived from a cancer cell line. In some embodiments, the neoplastic cells used in the method are human cells. In some embodiments, the neoplastic cells used in the method are mouse, rat, sheep, goat, pig, dog, cat, non-human primate, mammalian, or non-human mammalian cells. In some embodiments, the neoplastic cells are adherent cells.

In some embodiments, the method comprises contacting the decellularized biomatrix with neoplastic that are of the same species as the biomatrix. For example, in some embodiments, the method comprises providing a human decellularized biomatrix and contacting the human biomatrix with human neoplastic cells, or providing a rat decellularized biomatrix and contacting the rat biomatrix with rat neoplastic cells, and so on. In some embodiments, the method comprises providing a decellularized biomatrix and contacting the biomatrix with neoplastic cells that are of a different species than the biomatrix. For example, in some embodiments, the method comprises providing a rat decellularized biomatrix and contacting the rat biomatrix with human neoplastic cells, or providing a mouse decellularized biomatrix and contacting the mouse biomatrix with human neoplastic cells, or providing a mouse decellularized biomatrix and contacting the mouse biomatrix with rat neoplastic cells, and so on.

In some embodiments, the method comprises contacting the decellularized biomatrix with neoplastic that are of the same tissue of origin as the biomatrix. For example, in some embodiments, the method comprises providing a decellularized lung biomatrix and contacting the lung biomatrix with neoplastic lung cells, or providing a decellularized liver biomatrix and contacting the liver biomatrix with neoplastic liver cells, and so on. In some embodiments, the method comprises providing a decellularized biomatrix and contacting the biomatrix with neoplastic cells that are of a different tissue of origin than the biomatrix. For example, in some embodiments, the method comprises providing a decellularized lung biomatrix and contacting the lung biomatrix with neoplastic liver cells, or providing a decellularized kidney biomatrix and contacting the kidney biomatrix with neoplastic lung cells, or providing a decellularized biomatrix and contacting the mouse biomatrix with rat neoplastic cells, and so on.

In some embodiments, the method comprises contacting the decellularized biomatrix with neoplastic that are of the same species and of the same tissue of origin as the biomatrix. For example, in some embodiments, the method comprises providing a decellularized human lung biomatrix and contacting the human lung biomatrix with neoplastic human lung cells, for example, human lung cancer cells, or providing a decellularized rat liver biomatrix and contacting the rat liver biomatrix with neoplastic rat liver cells, and so on. In some embodiments, the method comprises providing a decellularized biomatrix and contacting the biomatrix with neoplastic cells that are of a different species, but of the same tissue of origin as the biomatrix. For example, in some embodiments, the method comprises providing a decellularized rat lung biomatrix and contacting the rat lung biomatrix with neoplastic human lung cells, or providing a decellularized mouse lung biomatrix and contacting the mouse lung biomatrix with neoplastic human lung cells, or providing a decellularized rat liver biomatrix and contacting the rat liver biomatrix with neoplastic mouse liver cells, and so on.

In some embodiments, the method comprises perfusing the decellularized biomatrix prior to, during, and/or after contacting the biomatrix with neoplastic cells. This can conveniently be done in a bioreactor as described herein, or by using any other suitable means for perfusing the biomatrix in a reactor, container, or vessel that can be used to expose the biomatrix to neoplastic cells. In some embodiments, the biomatrix is contacted with neoplastic cells by bathing the biomatrix in a culture media comprising the cells, or by flowing media comprising the cells over a surface of the biomatrix. In some embodiments, the biomatrix is contacted with neoplastic cells by injecting, flowing, infusing, perfusing, or otherwise contacting a biomatrix cavity with a suspension of the cells. For example, a lung biomatrix may be contacted with neoplastic cells by infusing a neoplastic cell suspension, e.g., a lung cancer cell suspension, into the bronchi via the trachea. Similarly, decellularized biomatrices of other organs may be contacted with neoplastic cells by infusing the cells into a fluid-carrying channel or vessel, whether effluent or influent, of the organ. Typically, the vessel used to contact the decellularized biomatrix with neoplastic cells is not a blood vessel, and preferably, contamination of the vascular space with neoplastic cells is avoided or minimized during the contacting, in order to retain a separation of the vascular and the tissue space of the biomatrix. This is of particular importance, if the tissue construct is intended for the measurement of the metastatic capabilities of the neoplastic cell population, which may involve assessing the potential of cells within the neoplastic cell population to migrate from the tissue space into the vascular space of the decellularized biomatrix.

In some embodiments, the method further comprises contacting the decellularized biomatrix with a neoplastic cell population and an additional, non-neoplastic cell population. The decellularized biomatrix can be contacted simultaneously with the neoplastic and the non-neoplastic cell populations together, for example, as a mixture of both cell types in the same medium, or sequentially, for example, the non-neoplastic cell population can be seeded first and then the neoplastic cell population, or vice versa. In some embodiments, the method comprises seeding both cell populations into the tissue space of the decellularized biomatrix, whereas, in other embodiments, the neoplastic cells are seeded into the tissue space and the non-neoplastic cells are seeded into the intravascular space. In some of the latter embodiments, the non-neoplastic cells are endothelial cells, or are of a cell type that can populate the blood vessel side of the basal membrane separating the tissue and the intravascular space of the decellularized biomatrix.

In some embodiments, the method comprises contacting the decellularized biomatrix with a non-neoplastic cell population that is origin-matched to the decellularized biomatrix, for example, non-neoplastic lung tissue cells, lung stromal cells, or lung epithelial cells may be seeded onto a decellularized lung biomatrix, non-neoplastic liver cells, hepatocytes, or fibroblasts may be seeded onto a liver biomatrix, and so on. Those of skill in the art will understand that these examples are non-limiting, and suitable, additional non-neoplastic cells and cell populations of the mentioned tissues and of other tissues as well as methods for obtaining such cell populations, including, for example, tissue homogenization or lavage, will be apparent to the skilled artisan.

### Bioreactors

Some aspects of this disclosure provide bioreactors for generating tissue constructs. In general, any suitable culture vessel can be used that allows perfusion of the decellularized biomatrix and/or contacting the biomatrix with neoplastic cells used for generating the tissue construct. This disclosure provides customized bioreactors for generating tissue constructs. Exemplary bioreactors are described in detail in the Example section. It will be apparent to those of skill in the art that the exemplified reactors are not limiting in any way, and that the reactors can be adapted to accommodate virtually any decellularized biomatrix and neoplastic cell type by the skilled artisan without more than routine experimentation.

In some embodiments, a bioreactor for generating and/or culturing an tissue construct is provided that comprises a decellularized biomatrix comprising a vascular space and a tissue space, a perfusion influx connected to the vascular space, a perfusion efflux connected to the vascular space, and a culture media influx connected to the tissue space of the biomatrix. In some embodiments, the decellularized biomatrix comprises an artery and a vein. In some embodiments, the perfusion influx is connected to an artery of the biomatrix, for example, to the pulmonary artery of a lung decellularized biomatrix. In some embodiments, the perfusion efflux is connected to a vein of the biomatrix, for example, a pulmonary vein through the left atrium of a lung biomatrix. In some embodiments, the biomatrix comprises an influent vessel connected to the tissue space of the biomatrix, and the culture media influx is connected to the epithelial space, for example, to the trachea of a lung biomatrix.

In some embodiments, the biomatrix comprised in the bioreactor is a lung biomatrix. In some such embodiments, the bioreactor does not comprise a ventilation loop. In some embodiments, the trachea of the lung biomatrix is used to infuse a neoplastic cell suspension into the tissue space of the lung biomatrix. In some embodiments, the lung biomatrix is perfused using the pulmonary artery as an influx and the pulmonary vein as an efflux.

In some embodiments, the bioreactor comprises a pump modulating the flow of perfusion fluid. In some embodiments, the pump is a peristaltic pump. In some embodiments, the bioreactor comprises a fluid reservoir holding a perfusion fluid. In some embodiments, the bioreactor comprises a fluid reservoir holding a neoplastic cell suspension. In some embodiments, the neoplastic cell suspension reservoir is positioned in a way that allows the cell suspension to contact the tissue space of the biomatrix by gravity flow. In some embodiments, the bioreactor comprises means to regulate the flow of perfusion fluid or culture media, for example, valves, stoppers, pumps, and so on.

### Methods for Evaluating and Analyzing Tissue Constructs

Some aspects of this disclosure provide methods and reagents for analyzing a tissue construct as described herein. In some embodiments, the analyzing comprises detecting a tumor nodule, quantifying a number of tumor nodules, measuring the size of the tumor nodules, detecting a level of gene expression associated with cancer, detecting a level of cell apoptosis or survival, detecting neoplastic cells in a liquid used for perfusing the tissue construct, and/or detecting a level of a signaling factor secreted by the neoplastic cells. Methods and reagents suitable for such analytical approaches are well known to those of skill in the art and include, but are not limited to, visual inspection of the tissue construct, e.g. via microscopy, immunohistochemistry (e.g., staining for proliferation markers or for proteins expressed within or on the surface of cells), TUNEL staining, BrdU staining, flow cytometry, protein and nucleic acid detection methods (e.g., PCR, RT-PCR, ELISA, etc.). Some suitable methods are described in more detail elsewhere herein, and additional methods will be apparent to the skilled artisan. The disclosure is not limited in this respect.

In some embodiments, the analyzing comprises determining the number of cells in the perfusion fluid used to perfuse a tissue construct as described herein. The number of cells in the perfusion fluid, also referred to as the number of circulating cells herein, can be used as an indicator for the metastatic properties of neoplastic cells cultured within the tissue construct. For example, in tissue constructs in which neoplastic cells are cultured within the interstitial space, and perfusion fluid is run through the vascular space, cells found in the perfusion fluid may stem from neoplastic cells that have migrated across the basement membrane separating the interstitial and the vascular space. Since the capability to migrate across the basement membrane and, thus, to enter into the blood stream, is a hallmark feature of metastasizing cancer cells, the ability to migrate across the basement membrane in a tissue construct is a biomarker useful for assessing metastatic capabilities of cells grown in tissue constructs provided herein.

The biomatrices, tissue constructs, tumor models, and methods provided herein allow for the isolation of at least three different cell populations, representing different phases of tumor growth and metastasis: (1) tumor growth; (2) circulating cells; and (3) metastasis or metastatic lesions. In some embodiments, the analyzing comprises isolating a cell or tissue from a tissue construct, or from medium used to perfuse a tissue construct. In some embodiments, the cell or tissue is isolated from a biomatrix seeded with neoplastic cells after a period of culturing the seeded biomatrix for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 21 days, at least 28 days, or at least a month. In some embodiments, the cell or tissue is isolated from a medium used to perfuse a biomatrix seeded with neoplastic cells after a period of culture (or after a period of perfusion) of the seeded biomatrix for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 21 days, at least 28 days, or at least a month. In some embodiments, the cell or tissue is isolated from an interstitial space not seeded with neoplastic cells, but perfused with a medium comprising circulating cells, e.g., circulating cells originating from an interstitial space seeded with neoplastic cells, after a period of culture (or after a period of perfusion) of the non-seeded interstitial space for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 21 days, at least 28 days, or at least a month.

In some embodiments, the analyzing comprises an assessment of one or more molecules secreted into a perfusion fluid by the cells cultured within the tissue construct. For example, in a lung tissue construct, the analyzing may comprise detecting a protein, peptide, or nucleic acid in a perfusion fluid run through the pulmonary artery. In some embodiments, the secreted factor assessed is a secreted factor associated with cancer, or with a property of neoplastic cells, for example, with the metastatic capabilities of neoplastic cells. Secreted factors associated with cancer are known to those of skill in the art and include factors that are typically secreted by a tumor, such as growth factors and pro-angiogenic factors, as are factors associated with the metastatic capabilities of neoplastic cells. Metastasis-associated factors include, for example, matrix metalloproteases (MMPs, e.g., MMP-1, MMP-2, MMP-3, MMP-9, and MMP-10, as well as other MMPs).

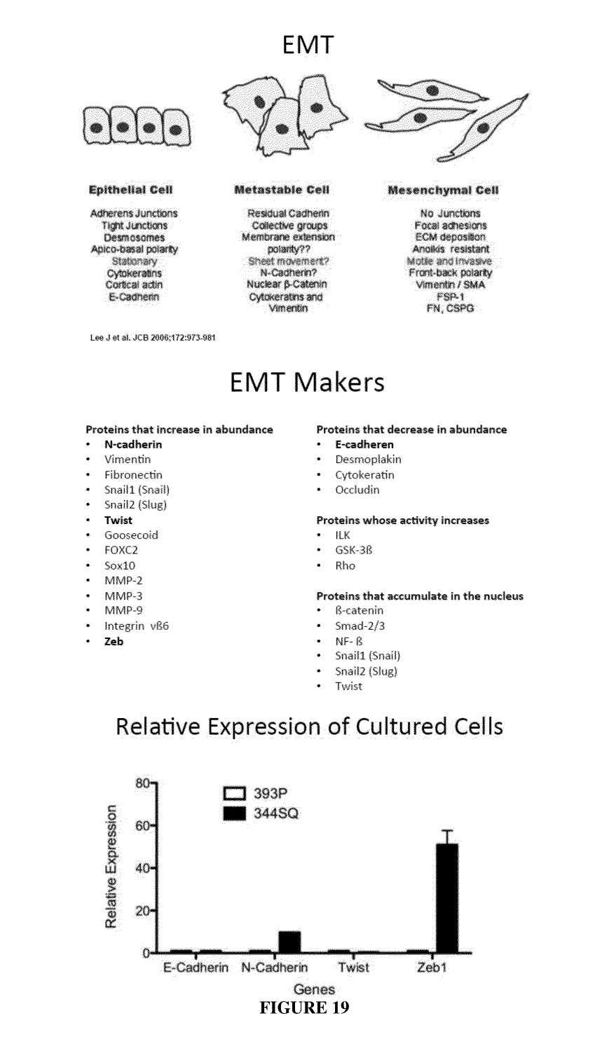

In some embodiments, the analyzing comprises assessing the expression of one or more gene products known to be regulated during epithelial-mesenchymal transition (EMT), another hallmark of some metastasizing cancers. Genes and gene products implicated in EMT are well known to those of skill in the art and some exemplary gene products and their regulation during EMT are described in FIG. 19. In some embodiments, the analyzing comprises establishing a baseline value for the expression of a gene or gene product, for example, a quantitative baseline value representing a non-metastatic state, and comparing an assessed value in a tissue construct to that baseline value. A deviation of the measured value from the baseline value that is consistent with EMT may indicate an increased metastatic potential of the cells cultured within the tissue construct.

Methods for Identifying Agents having Anti-Cancer Properties

Some aspects of this invention provide methods and reagents for identifying an anti-cancer agent using a tissue construct as provided herein. In some embodiments, the method comprises contacting a tissue construct as provided herein with candidate agent, for example, by adding the agent to a perfusion fluid and perfusing the construct for a time sufficient for the agent to contact the neoplastic cells in the construct. In some embodiments, the method comprises assessing a biomarker associated with cancer in the tissue construct contacted with the candidate agent. In some embodiments, the method comprises comparing the assessed biomarker with a reference value. In some embodiments, the method comprises identifying the candidate agent as an anti-cancer agent if the assessment of the biomarker associated with cancer in the construct contacted with the candidate agent shows that a neoplastic or cancer characteristic of the tissue construct is absent or diminished as compared to the reference value.

In some embodiments, the biomarker associated with cancer comprises cell proliferation, cell survival, tumor formation, tumor number, tumor growth, tumor volume, tumor phenotype, tumor nodule formation, tumor nodule number, tumor nodule growth, tumor nodule structure, tumor nodule volume, tumor nodule phenotype, expression of a gene product, expression of an oncogene, repression of a tumor suppressor, presence or abundance of neoplastic cells in a perfusion efflux fluid, expression of mesenchymal markers by cells present in a perfusion fluid, and/or a metastatic activity of cells present in a perfusion fluid. In some embodiments, the reference value is a value observed or expected in a tissue construct not contacted with a candidate agent

In some embodiments, the tissue construct comprises neoplastic cells known or suspected to metastasize, and the biomarker assessed is a metastasis-associated biomarker, for example, the number of circulating cells, or the expression of a gene product or a level of expression of a gene product consistent with EMT. In embodiments, if a decreased number of circulating cells is found in the tissue construct treated with the candidate compound than observed or expected in an untreated control construct, or if expression of a gene product consistent with EMT is not found, or found at a lower level than in an untreated control construct, then the candidate compound is determined to have anti-cancer, or anti-metastatic properties.

In some embodiments, the candidate compound is a small molecule compound. In some embodiments, the method is used to screen a library of candidate agents, for example, a library of chemical compounds. In some embodiments, the candidate agent comprises a nucleic acid molecule, for example, a DNA molecule, an RNA molecule, or a DNA/RNA hybrid molecule, single-stranded, or double-stranded. In some embodiments, the candidate agent comprises an RNAi agent, for example, an antisense-RNA, an siRNA, an shRNA, a snoRNA, a microRNA (miRNA), or a small temporal RNA (stRNA). In some embodiments, the candidate agent comprises an aptamer. In some embodiments, the candidate agent comprises a protein or peptide. In some embodiments, the candidate agent comprises an antibody or an antigen-binding antibody fragment, e.g., a F(ab′)₂ fragment, a Fab fragment, a Fab′ fragment, or an scFv fragment. In some embodiments, the antibody is a single domain antibody. In some embodiments, the agent comprises a ligand- or receptor-binding protein.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Materials and Methods

All the animal experiments were carried out in accordance with all applicable laws, regulations, guidelines, and policies governing the use of laboratory animals in research. The protocols for animal experiments were approved by the Institutional Animal Care and Use Committee at the Methodist Hospital Research Institute.

### Rat Lung Isolation

Six-week to 12-week-old male Sprague-Dawley rats were anesthetized with ketamine (100 mg/kg) and xylazine (10 mg/kg). After 5 to 10 minutes, once rats were anesthetized, they were shaved on the chest and abdomen, the skin was treated with povidone-iodine topical antiseptic (Betadine), and a bilateral thoracotomy was performed to open the thoracic cavity. Two mL of heparin were injected (1,000 U/mL, Sagent Pharmaceuticals, Schaumburg, Ill.) into the right ventricle of the beating heart to prevent formation of blood clots in the lung. Next, the rib cage was removed and 20 mL of heparinized phosphate-buffered saline injected (12.5 U/mL; heparinized PBS) in the right ventricle after placing an 18-gauge needle (Cotran, Portsmouth, R.I.) in the left ventricle as a vent. The superior vena cava and inferior vena cava were cut, and the lungs were flushed again with 20 mL of heparinized PBS. Next, the trachea was divided at the level of the thyroid, the branches of the aorta at the arch, and the descending aorta at the level of the hemiazygos vein. The heart-lung block was then separated away from the esophagus and the rest of the rat body. A ventriculotomy was performed to expose the right and left ventricles and a custom-made prefilled 18-gauge stainless steel needle (Cotran) was fitted through the right ventricle into the main pulmonary artery. This was secured with a 2-0 silk tie (Ethicon, San Angelo, Tex.).

A female Luer bulkhead (Cole-Parmer, Vernon Hills, Ill.) was placed in the left ventricle and secured it with a 2-0 silk tie. The pulmonary artery cannula was flushed with heparinized PBS and placed it in a 50-mL tube containing heparinized PBS.

### Lung Decellularization

A simple decellularization chamber was designed (FIG. 1A) to remove the native rat cells from the lung. The decellularization chamber was created from a 500-mL glass bottle (Fisher Scientific, Inc, Suwanee, Ga.). Two holes were drilled into the cap with a ⅛-inch adapter drill bit, fitted the female Luer bulkhead into the hole, and secured it with a black nylon ring (Cole-Parmer). One of the Luer sides was connected to a small length of flexible plastic tubing (Tygon; Cole-Parmer) touching the bottom surface of the bottle for outflow. This chamber was connected to a 2-foot length of Tygon tube with a male Luer lock (Cole-Parmer), which was then connected by means of a 6-inch Masterflex roller tube (Cole-Parmer) to a female Luer bulkhead going into a beaker to collect the outflow of the bottle. All these items were autoclaved. The pulmonary artery cannula was connected to the cap of the decellularization chamber. A pierced capped 500-mL bottle with a primary intravenous set (Hospira, Lake Forest, Ill.) was used to introduce different solutions through the pulmonary artery by means of a cannula at physiologic perfusion pressure (FIG. 1A).

Heparinized PBS ran for 15 minutes through the pulmonary artery at a perfusion pressure of 30 mm Hg for the initial wash and then 0.1% sodium dodecyl sulfate (Fisher Scientific) in deionized water was perfused through the lung for 2 hours for decellularization. After decellularization, deionized autoclaved water was perfused through the lung scaffold for 15 minutes, followed by 1% Triton-X-100 (Fisher Scientific) in deionized water for 10 minutes. Next, the tubing that was going to the beaker was attached to the inflow Luer adapter of the bottle containing the hanging lung, and the perfusion system was connected to the Masterflex pump using PharMed BPT tubing (Cole-Parmer), Luerlock connectors, and Tygon tubing to remove the excess sodium dodecyl sulfate with autoclaved PBS containing antibiotic (100 IU/mL penicillin, 100 μg/mL streptomycin, and 0.25 μg/mL amphotericin; MP Biomedicals, Solon, Ohio). Lungs were perfused for 72 hours and frozen at −80° C., if not used immediately.

### Cell Culture

The human alveolar basal epithelial adenocarcinoma cell line A549 was supplied by Dr Kurie's laboratory (The University of Texas MD Anderson Cancer Center, Houston, Tex.). Lung cancer cell lines H460 and H1299 were supplied by Dr Haifa Shen's laboratory (The Methodist Hospital Research Institute, Houston, Tex.). These cell lines were grown in BD T175 cell culture flasks in complete medium made from Roswell Park Memorial Institute (RPMI) 1640 medium (Hyclone, South Logan, Utah) supplemented with 10% fetal bovine serum (Lonza, Walkersville, Md.) and antibiotics (100 IU/mL penicillin, 100 μg/mL streptomycin, and 0.25 μg/mL amphotericin; MP Biomedicals) at 37° C. in 5% CO2. Once cells were 85% confluent, they were washed with PBS and subjected to trypsinization using 0.25% trypsin (Cellgro, Manassas, Va.) to collect the cells from flasks. Cells were washed with medium and finally suspended in 30 to 50 mL of serum-free medium. Approximately 50 million cells were used for seeding the lung biomatrix.

### Bioreactor

A simplified, small, closed-system bioreactor was set up in an incubator for lung cell culture on the lung biomatrix (FIG. 1B). A custom-designed 500-mL glass bottle was used with three holes in the cap fitted with a female Luer thread-style panel (Cole-Parmer), one for the pulmonary artery cannula, one for the trachea cannula, and one for circulation of medium from the bottle. Medium was constantly circulated with the help of a Masterflex pump (Cole-Parmer) through a 10-foot length of silicone oxygenator tubing wrapped around a mesh of wire solenoid (Cole-Parmer). The medium was perfused through the pulmonary artery cannula into lungs at a flow rate of 6 mL/min. For controlled flow through the pulmonary artery, it was connected to a three-way stopcock (Smith Medical, Dublin, Ohio). The bottle was filled with 150 mL of complete medium or serum-free medium, which was circulated through the oxygenator tubing to prevent air bubbles.

Before seeding the human lung cancer cells into the lung biomatrix, the trachea was cannulated using an 18-gauge needle, and the scaffold was fixed to the bioreactor bottle in a hanging position; the complete medium was perfused through the lung biomatrix for 30 minutes at 37° C. in 5% CO2 at a rate of 6 mL/min using a roller pump. Afterward, the 50 million cells suspended in 30 to 50 mL of medium were seeded into the lungs through the tracheal cannula using a sterile syringe fed by gravity.

The bioreactor was placed in the incubator for 2 hours to allow for attachment of the cells. After 2 hours the scaffold was perfused at a flow rate of 6 mL/min. The medium in the bottle (approximately 100 to 200 mL) was changed every 1 to 2 days to make sure the nutrients were optimal for cell growth. The cells were grown on the biomatrix for 7 to 14 days. The lung biomatrix was then carefully removed from the bioreactor bottle, maintaining sterile conditions, and a lobectomy was performed under the culture hood by tying the anatomic lobe with 2-0 silk and resecting it on different days. The experiments were repeated at least three times.

### DNA Extraction

DNA was extracted from three native rat lungs and three decellularized rat lungs using Qiagen DNeasy DNA isolation kit (Qiagen, Valencia, Calif.). Equal amounts of tissues (20 mg) were taken, minced into small pieces with a surgical blade, and digested overnight with proteinase K in ATL buffer provided with the kit. After complete digestion, AL buffer and 100% ethanol were added and the mixtures loaded on columns. The mixtures were subjected to centrifugation, washed per the manufacturer's instructions, and finally eluted in 100 μL of elution buffer. DNA concentration was quantified by using a Nanodrop ND 1000 spectrophotometer (Thermo Fisher Scientific, Wilmington, Del.).

### Histology

Lobes of same lungs were dissected at day 0 (day of seeding cancer cells onto biomatrix), day 3, day 7, and day 11 or day 14 (or both) in sterile conditions under the culture hood to see the progression of tumor growth. After lobectomy, lung tissues were placed in 70% ethanol and analyzed in the Pathology Core Laboratory at The Methodist Hospital Research Institute. Briefly, the tissues were fixed in 10% formalin overnight, processed, and embedded in paraffin. Embedded tissues were cut into 4-1 μm slices and mounted on slides, and the paraffin was removed; antigen retrieval was performed with antigen unmasking solution (H-3300; Vector Laboratories, Burlingame, Calif.) in a steamer for 20 minutes. Slides were cooled for 20 minutes at room temperature, washed in PBS, and stained with hematoxylin and eosin, Movat Pentachrome (American MasterTech Scientific, Lodi, Calif.), elastin (VVG kit, Ventana Nexus, Tucson, Ariz.), and other markers following the standard protocol [12]. Stained slides were examined by expert board-certified pathologists, and images were captured using a microscope (Olympus, Center Valley, Pa.).

### Example 1

### Tissue Constructs Comprising Rat Lung Biomatrix

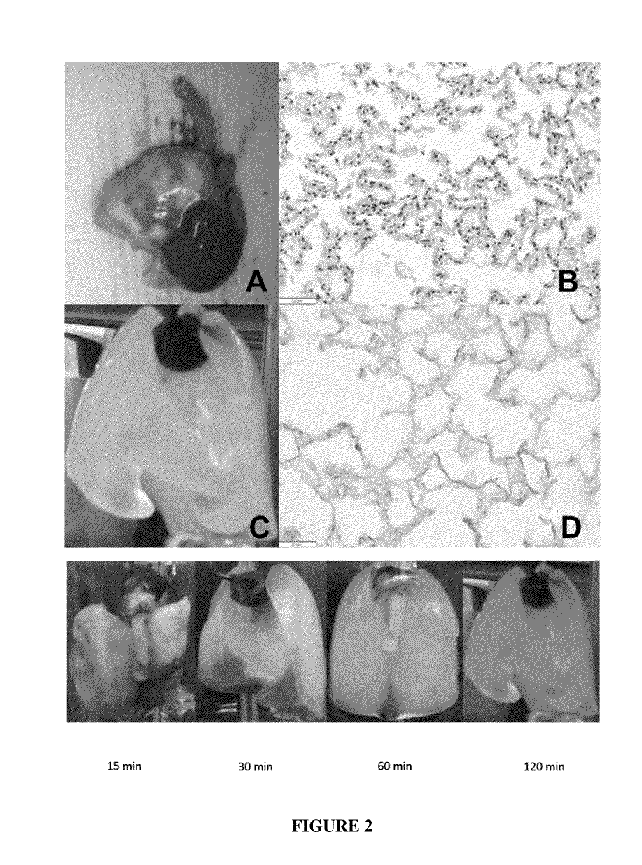

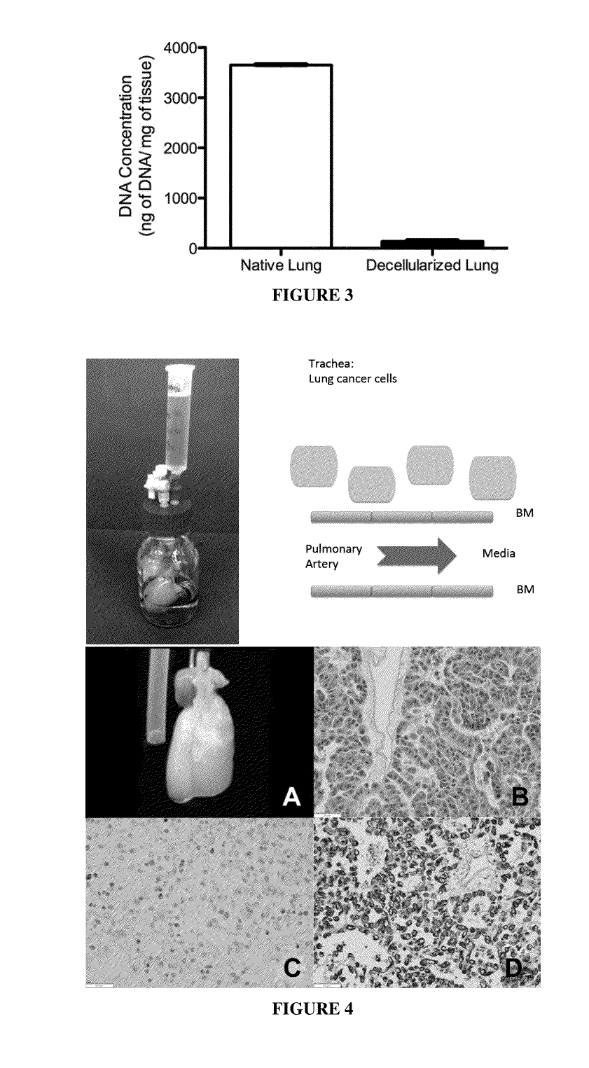

The native heart-lung block was harvested from adult rats (FIG. 2A). On perfusion with heparinized PBS through a cannulated pulmonary artery, flow exited the left ventricle without leakage, suggesting preservation of an intact vasculature. Hematoxylin and eosin staining of the native lung exhibited normally cellularized alveoli with pneumocytes and endothelial cells in the interstitial vessels (FIG. 2B). Using the custom-made decellularization chamber, it was possible to remove most cells in the rat lung (FIG. 2C). Hematoxylin and eosin (FIG. 2D) and Movat Pentachrome staining showed no rat cells present in the scaffold. Movat Pentachrome and elastin staining showed the presence of preserved biomatrix composed of collagen, elastin, and proteoglycans as well as an elastic fiber network of septal, axial, and pleural fibers of the airway and alveoli remaining intact. The DNA concentration of the decellularized lung was reduced to less than 5% of that of native lung (FIG. 3).

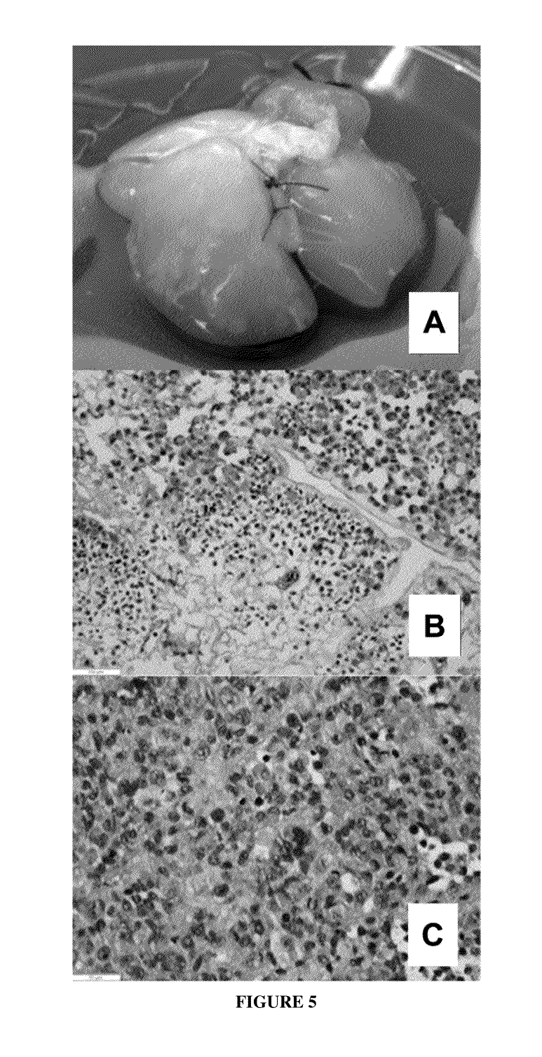

All three human lung cancer cell lines (A549, H1299, and H460) engrafted onto the rat biomatrix in the custom-made bioreactor and created perfusable tumor nodules. A549 cells grown on the scaffold produced no nodules on day 3, but by day 11 (FIG. 4A) solid tumor nodules had formed. Hematoxylin and eosin staining on day 3 showed cells attaching to the biomatrix in airways, terminal bronchioles, alveolar ducts, and alveoli with intact vasculature (FIG. 4B). The cells grew along the basement membrane of the alveoli.

Hematoxylin and eosin staining on day 11 showed most of the scaffold covered in the area of the nodule with a lack of organized growth of tumor cells along the basement membrane. The scaffold was populated with A549 cells (derived from a lung adenocarcinoma [10]) and had features similar to human bronchioloalveolar pattern carcinoma. The cells stained for the epithelial marker CK7 and the lung-specific nuclear marker TTF-1, with a high frequency of the proliferation marker Ki-67 (FIG. 4C). The cells also stained for vimentin (FIG. 4D), β-catenin, and E-cadherin.

H460 cells grown on the scaffold produced numerous nodules on the biomatrix after 7 days (FIG. 5A). Hematoxylin and eosin staining of H460 cells, which were derived from the pleural fluid of a patient with large cell lung cancer, showed features of poorly differentiated non-small cell lung cancer with sheetlike growth of polygonal cells along the airways and alveoli with intact vasculature (FIGS. 5B, 5C). Two distinct patterns were seen in the biomatrix: areas of numerous mitoses to areas of apoptotic cells with pyknotic nuclei (FIG. 5B).

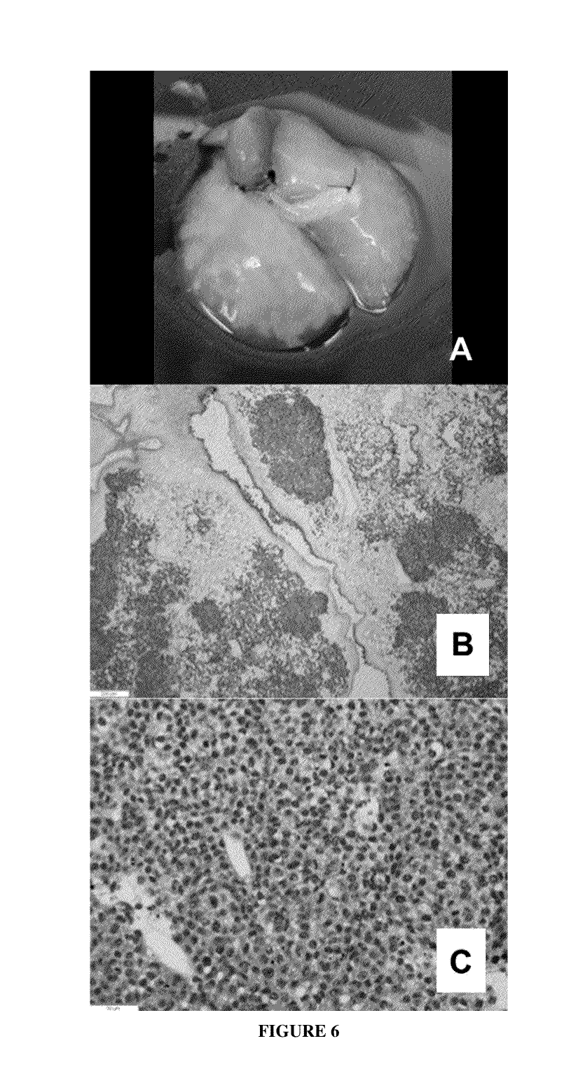

H1299 cells also produced numerous nodules on the biomatrix after 7 days (FIG. 6A). These cells, which were derived from a lymph node metastasis of a patient with non-small cell lung cancer, grew well on the scaffold. Hematoxylin and eosin staining showed very poorly differentiated features with malignant cells growing in a disorganized fashion with intact vasculature. The disordered growth resembled metastatic disease more than that of the other cell lines, which showed greater interaction with the biomatrix (FIGS. 6B, 6C).

### Example 2

### Comparison of Neoplastic Cells in 2D Culture and Cultured within Tissue Constructs

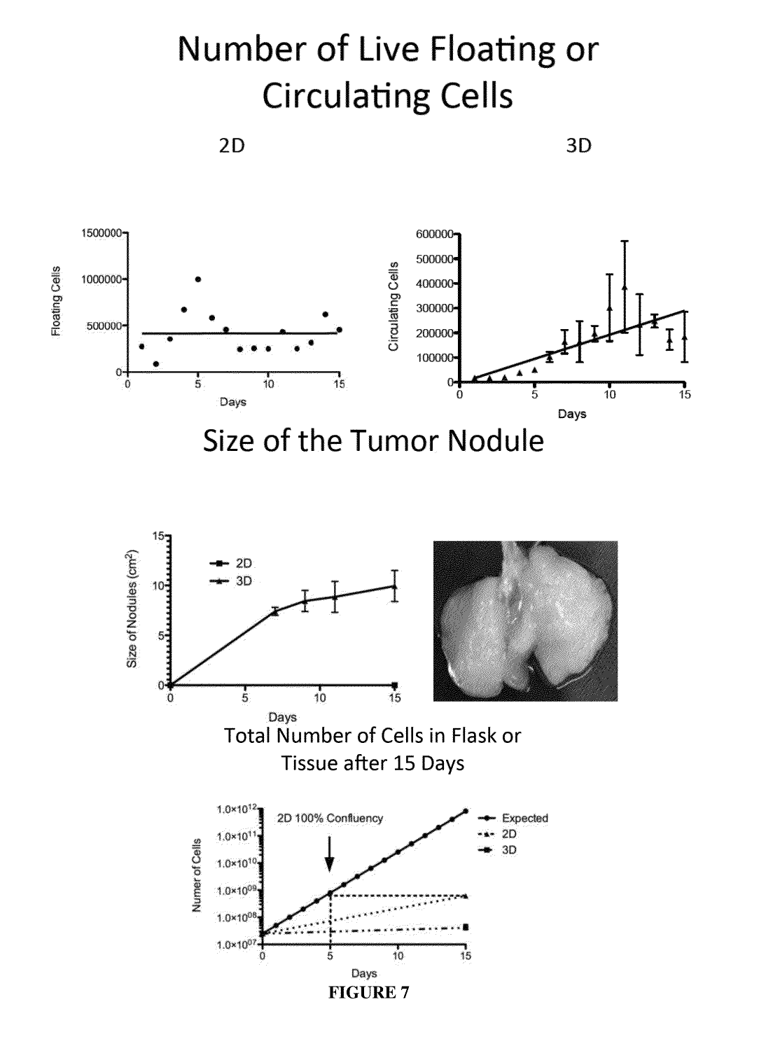

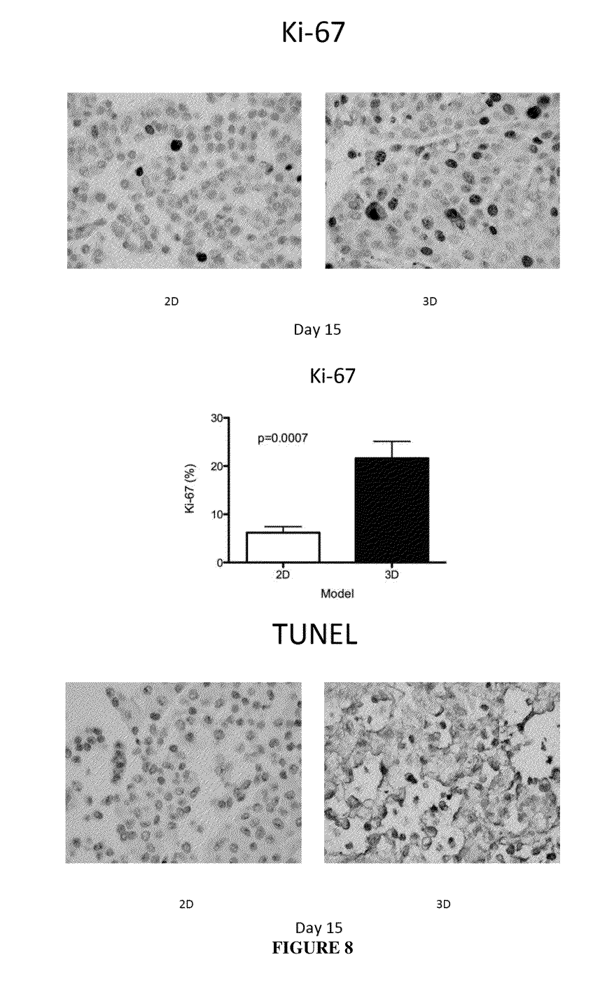

Conventional cell culture in a culture dish was compared to culture within a tissue construct over a time period of 15 days. The number of live floating cells was determined in a conventional culture dish culture (2D culture) of A549 cells, and compared to the number of circulating cells in a tissue construct comprising A549 cells (3D culture, FIG. 7, upper panel). In contrast to the 2D culture, in which the number of floating cells remained constant over a time period of 15 days, the number of circulating cells in 3D culture increased over time. The size of tumor nodules was also found to increase over time in 3D culture, whereas no tumor nodules were formed in 2D culture (FIG. 7, middle panel). The total number of cells was decreased in 3D culture as compared to 2D culture. The proliferation marker Ki-67 was expressed in a significantly larger portion (about 20%) of cells in 3D culture as compared to cells in 2D culture (about 6%, FIG. 8, upper and middle panel), and the 3D culture showed a complex pattern of TUNEL-stained cells, whereas no such staining was observed in cells in 2D culture.

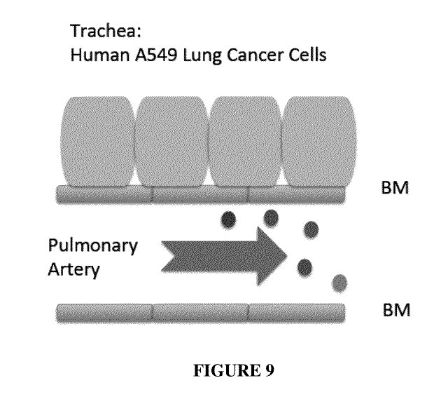

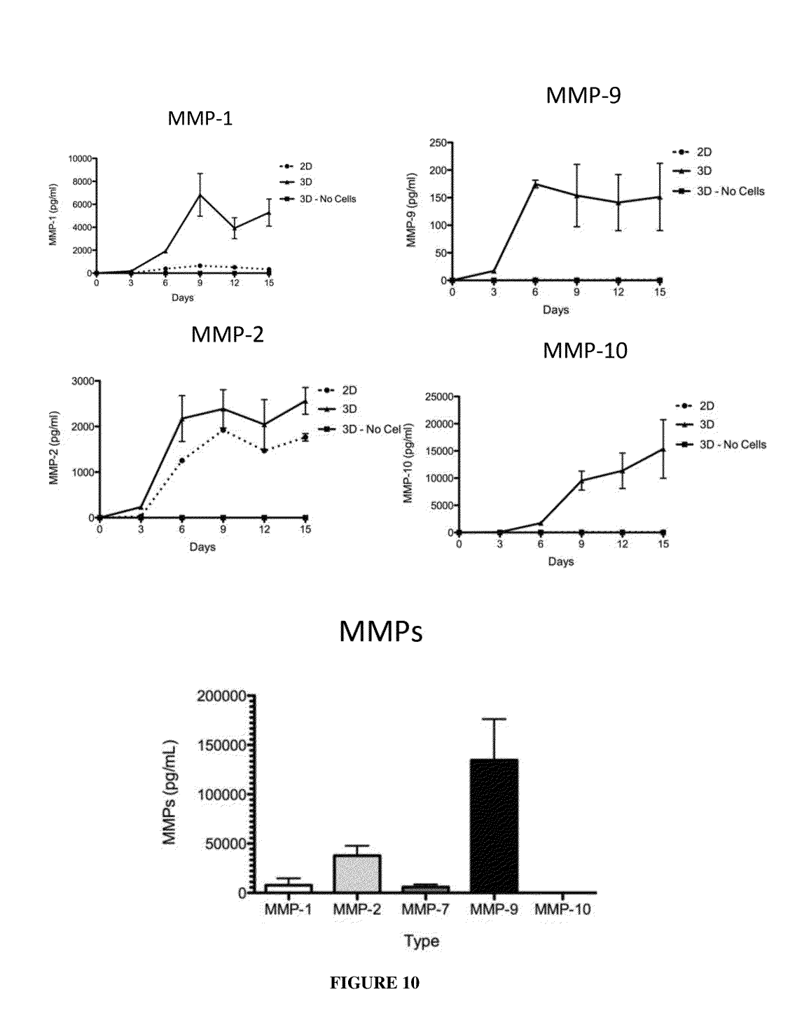

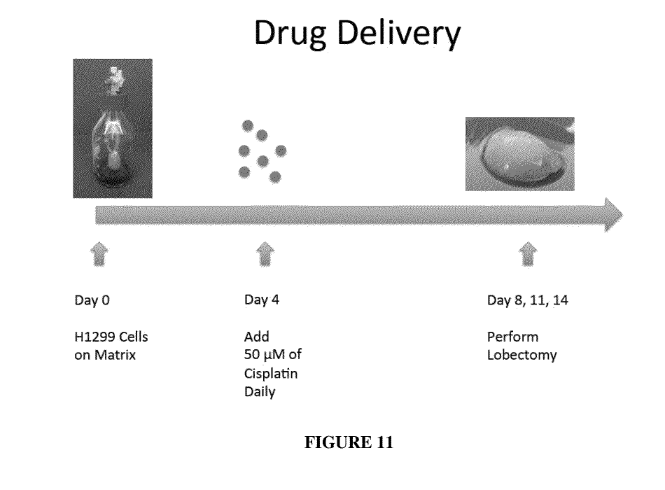

MMPs secreted into the perfusion fluid of tissue constructs in which human A549 lung cancer cells were grown were assessed (FIG. 9), and compared to MMPs secreted into the culture supernatant of the same cells in 2D culture. Levels of MMPs 1, 2, 9, and 10 were assessed. Cells grown in 3D culture expressed and secreted all four MMPs at high levels, whereas cells grown in 2D culture did not express MMP-9 and expressed low levels of MMP 1 and MMP 10 compared to those observed in 3D culture. There was no difference in MMP 2 production between the 2D culture and 3D culture.

### Example 3

### Tissue Constructs as a Model for Evaluation of Treatment

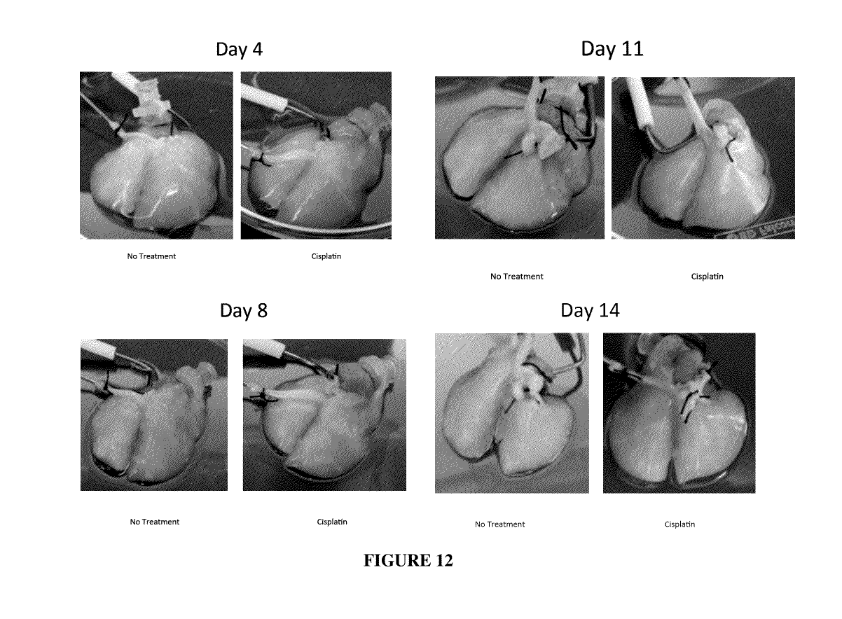

Tissue constructs were generated by culturing human 1299 cells, also known as NCI-H1299 or CRL-5803, a human non-small cell lung carcinoma cell line, within rat decellularized biomatrices. Treatment (Tx) with 50 μM Cisplatin was commenced at day 4 of culture, and treated tissue constructs and untreated controls were assessed and compared at days 4, 8, 11, and 14 of culture (FIGS. 11 and 12). At days 8 and 11, lobectomy of the right upper lobe (RUL), and the right middle lobe (RML), respectively, were performed for tissue assessment.

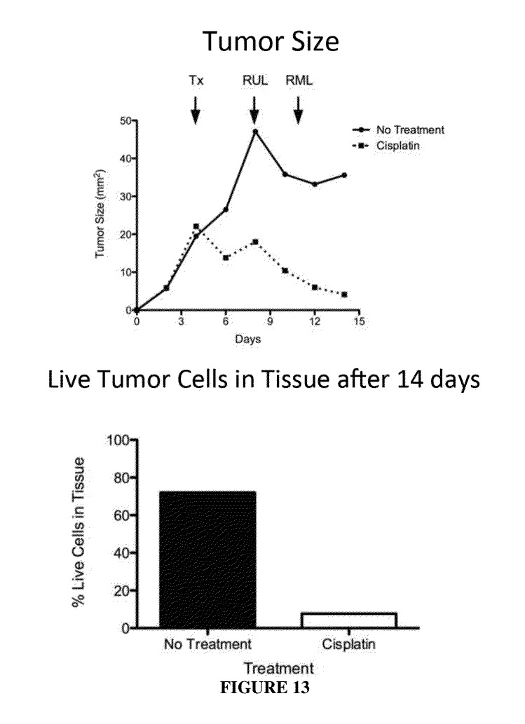

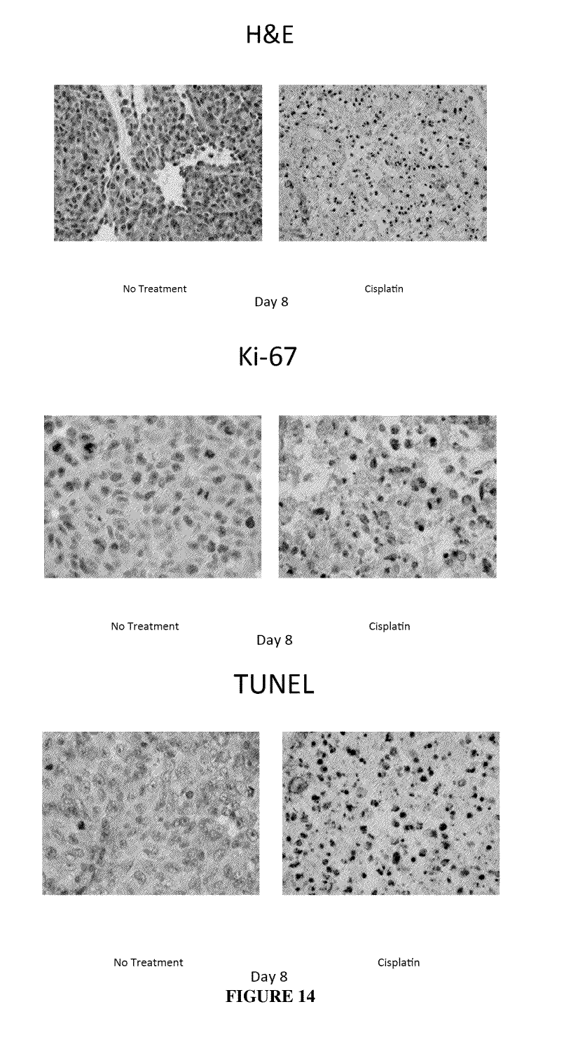

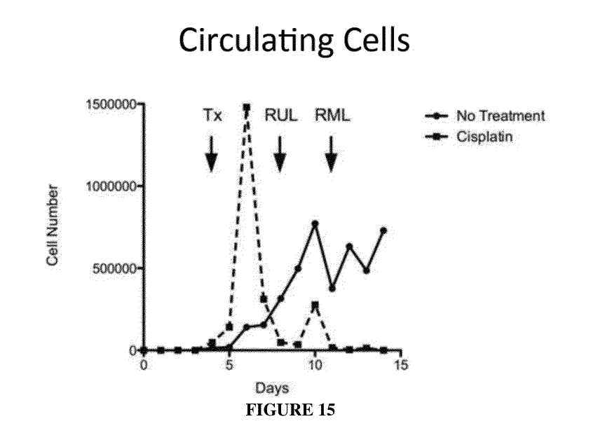

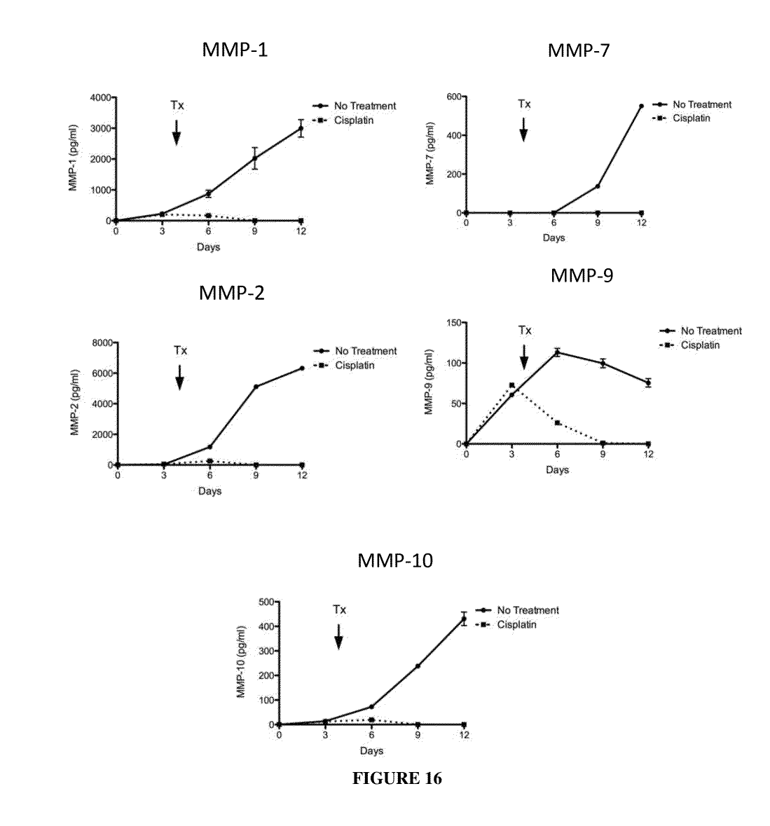

Tumor size was dramatically decreased in Cisplatin-treated tissue constructs as compared to about their untreated counterparts, and so was the percentage of live cells in the treated tissue constructs as compared to the non-treated constructs (FIG. 13). Ki-67 expression was decreased in treated constructs and TUNEL staining revealed widespread apoptotic processes in the treated, but not in the untreated tissue constructs (FIG. 14). An assessment of circulating cells revealed that while untreated tissue constructs showed a steady increase in the number of circulating cells, treated constructs showed a gradual decrease over the observed time period after initial rapid release of the circulating cells (FIG. 15). Treatment also affected the secretion of MMPs into the perfusion fluid (FIG. 16). Levels of MMPs 1, 2, 7, 9, and 10, all gradually increased over time in the untreated tissue constructs. In the treated tissue constructs, however, MMP levels increased only until treatment was started, at which point levels of all MMPs were diminished. At the end of the observation period, no detectable levels of any of the assessed MMPs were present in the treated tissue constructs.

### Example 4

### Tissue Constructs as a Model for Evaluation of Metastasizing Potential

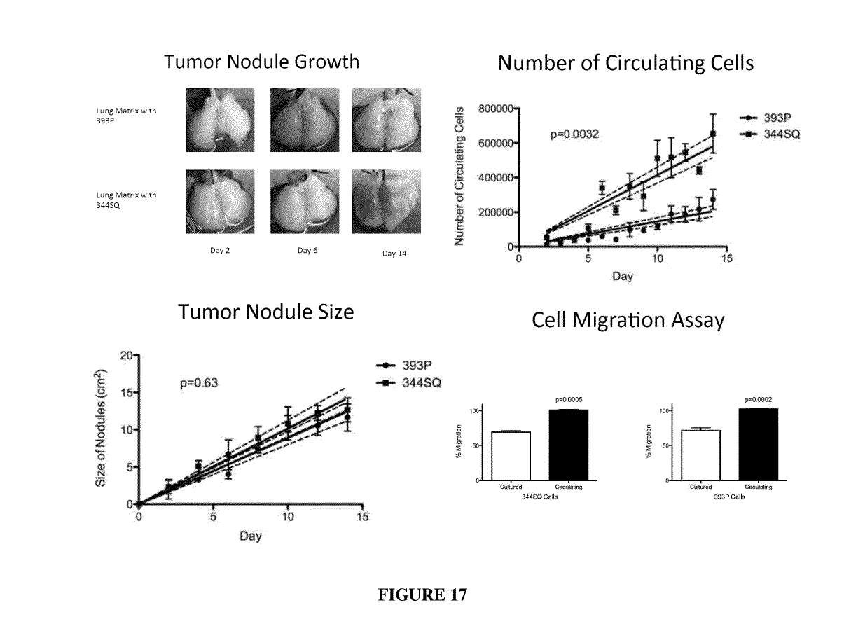

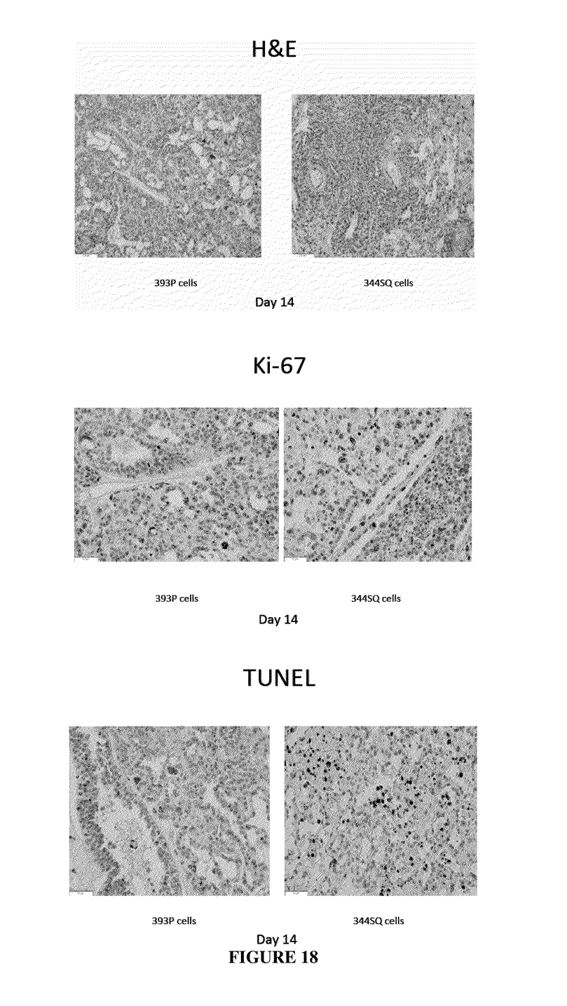

In order to determine whether differences in metastatic potential of neoplastic cells can readily be assessed in tissue constructs as provided herein, tissue constructs comprising non-metastatic 393P lung adenocarcinoma cells were compared to tissue constructs comprising metastatic 344SQ lung adenocarcinoma cells. FIG. 17 shows a visual assessment of these constructs at days 2, 6, and 14 (upper left panel). The number of circulating cells increased gradually in both types of tissue constructs, but the number of circulating cells was significantly higher, and increased at a greater rate in the constructs comprising metastatic 344SQ lung adenocarcinoma cells (upper right panel). Tumor nodule size was almost identical in both types of tissue constructs (lower left panel). The migratory ability of circulating and cultured 344SQ and 393P cells was compared using a Boyden chamber cell migration assay. In both cell types, the circulating tumor cells had a greater ability to migrate as compared to the cultured cells (lower right panel), suggesting that circulating tumor cells have a greater ability to migrate and to metastasize as compared to the cultured tumor cells. H&E stainings, Ki-67 stainings, and TUNEL stainings of lung tissue constructs comprising non-metastatic 393P cells and metastatic 344SQ cells are shown in FIG. 18.

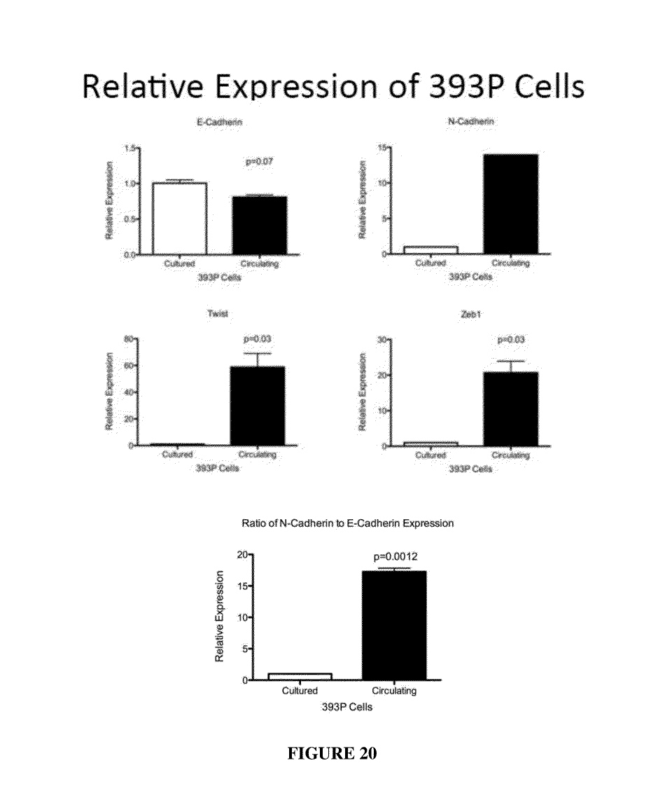

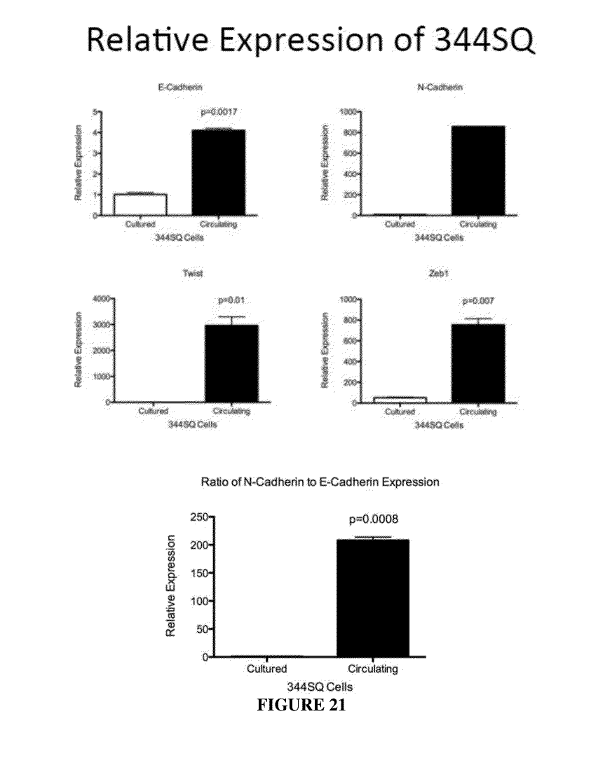

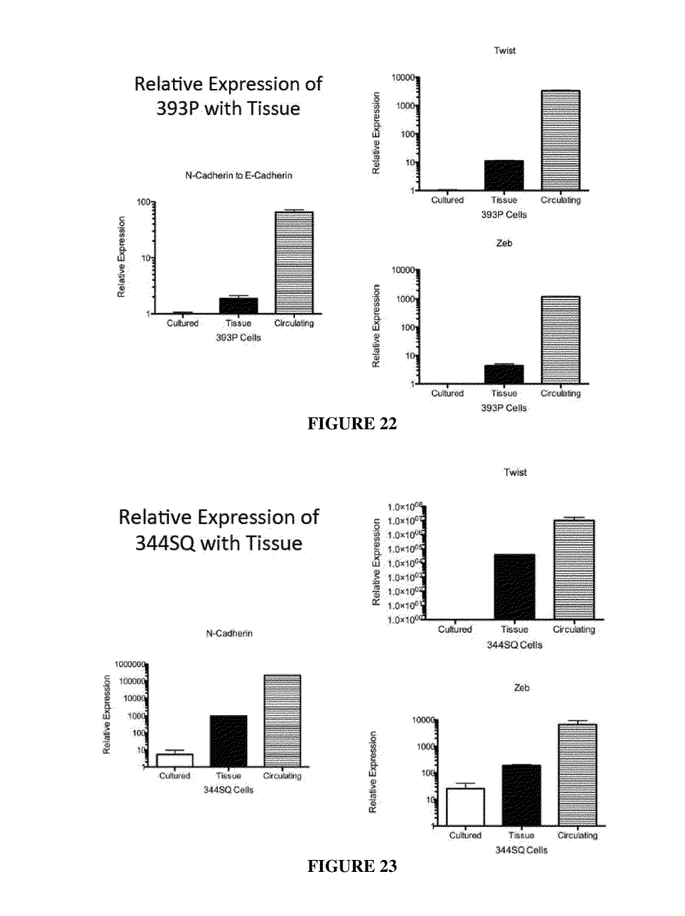

FIG. 19 shows an overview over epithelial-mesenchymal transition (EMT, upper panel), some exemplary marker proteins that are regulated during EMT (middle panel), and a comparison of exemplary marker protein expression in 393P and 344SQ cells (lower panel). FIG. 20 shows a comparison of EMT marker expression in 393P cells in 2D culture (“cultured”) to expression in cells recovered from perfusion fluid of tissue constructs comprising decellularized lung biomatrix and 393P cells (“circulating”). FIG. 21 shows a comparison of EMT marker expression in 344SQ cells in 2D culture (“cultured”) to expression in cells recovered from perfusion fluid of tissue constructs comprising decellularized lung biomatrix and 344SQ cells (“circulating”). FIG. 22 shows a comparison of EMT marker expression in 393P cells in 2D culture (“cultured”) to expression in cells recovered from the tissue or the perfusion fluid of tissue constructs comprising decellularized lung biomatrix and 393P cells (“tissue,” and “circulating,” respectively). FIG. 23 shows a comparison of EMT marker expression in 344SQ cells in 2D culture (“cultured”) to expression in cells recovered from perfusion fluid of tissue constructs comprising decellularized lung biomatrix and 344SQ cells (“circulating”).

### Example 5

### Tissue Constructs as a Model for Metastasis

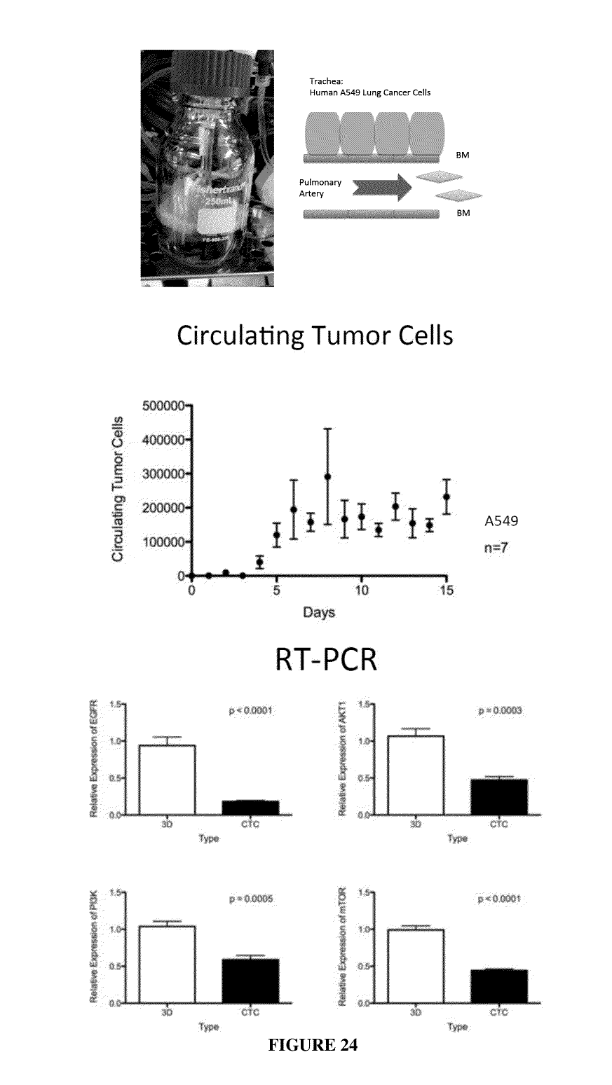

FIG. 24 shows a bioreactor harboring a lung tissue construct comprising human A549 lung cancer cells seeded into the interstitial space of a decellularized lung biomatrix via the trachea. Media was run through the vascular space via the pulmonary artery. The A549 cells formed nodules in the interstitial space and some of the cells migrated across the basement membrane (BM) and into the vascular space. These cells, referred to as circulating cells, are represented by a diamond in the schematic of the upper panel of FIG. 24. The number of circulating cells was monitored daily over a time period of 15 days in 7 different tissue constructs seeded with A549 lung cancer cells. As shown in the middle panel of FIG. 24, while virtually no circulating cells were observed in the first three days after seeding, increasing numbers were observed starting on day 4. Circulating A549 cells were isolated from medium used to perfuse a tissue construct seeded with A549 cells, and the gene expression of the circulating cells (“CTC”) was profiled and compared to that of A549 cells grown in the interstitial space of the tissue construct (“3D”). RNA was obtained from circulating A549 cells and subjected to microarray and RT-PCR gene expression analysis. Significant differences in gene expression levels were detected between circulating and interstitial cells, as illustrated by the RT-PCR data for the expression levels of some exemplary genes, EGFR, ALK1, P13K, and mTOR in circulating cells and interstitial cells, shown in the lower panel of FIG. 24. These results demonstrate that the tissue constructs provided herein are useful for isolating and analyzing circulating cells that have migrated across the basal membrane of a biomatrix.

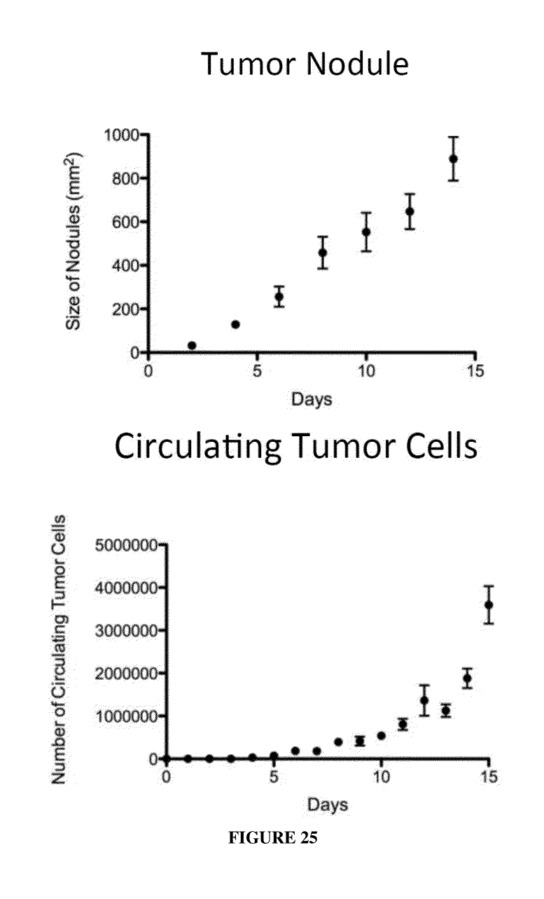

FIG. 25 shows measurements of tumor nodule size and number of circulating cells in tissue constructs seeded with cells of a different cancer cell line, NIH-H1299. Similar to the observations from tissue constructs seeded with A549 cells, both nodule size and number of circulating cells were observed to increase over time. While nodules could be detected as early as day 2 after seeding, there were virtually no circulating cells observed until day 4.

As explained above, circulating cells represent cells with an increased ability to migrate and to metastasize as compared to the cells that populate the interstitial space of a tissue construct after seeding. The observations illustrated in FIGS. 24 and 25 are consistent with the notion that circulating cells represent cells that have undergone an epithelial-mesenchymal transition (EMT), a hallmark of tumor invasiveness. Without wishing to be bound by theory, the data obtained supports the view that the process of EMT and the subsequent transgression of the biomatrix basement membrane takes a couple of days for the seeded cells to complete.

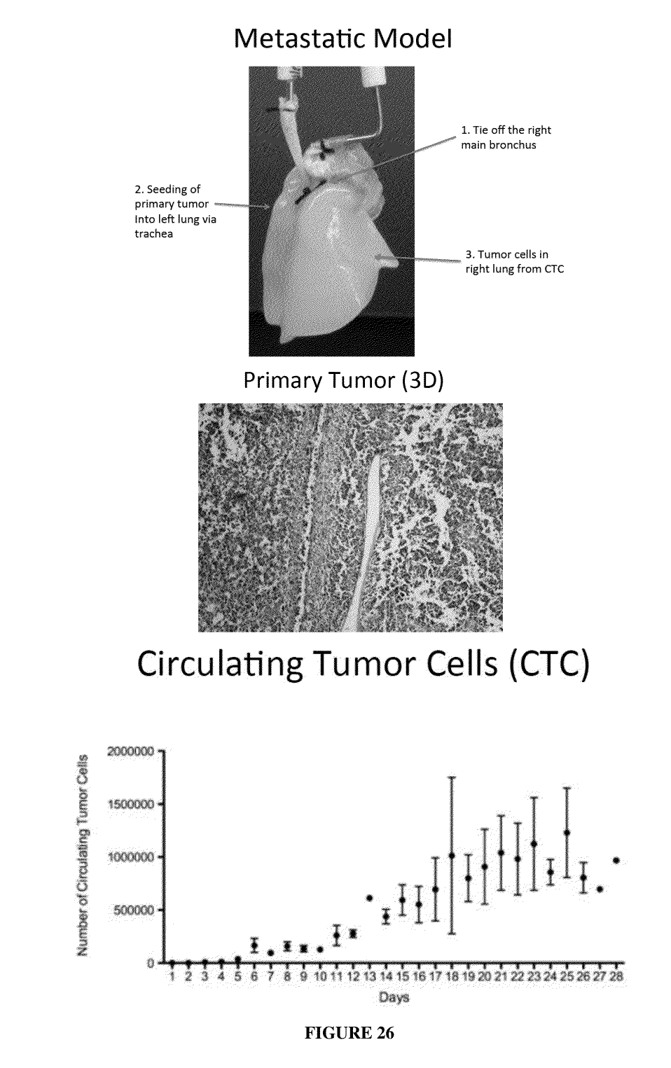

In order to investigate the formation of metastatic lesions originated from circulating cells, a metastatic tumor model was developed (FIG. 26, upper panel). A decellularized lung biomatrix was seeded with lung cancer cells (e.g., A549, 344SQ, or NIH-H1299 cells) after one bronchus was tied off to prevent influx from the trachea into that bronchus. Cancer cells were seeded into the decellularized lung biomatrix via infusion through the trachea, as described in more detail elsewhere herein. Because one bronchus was tied off, no cells were able to reach the interstitial space of that bronchus. Accordingly, all cells were seeded into the interstitial space of the “open” bronchus. Culture of the cells in the tissue construct was as previously described, with medium being flown through the vascular space of both bronchi via the pulmonary artery. Medium flow to the tied-off bronchus was not impaired. The seeded cancer cells were cultured in the interstitial space of the open bronchus, forming primary tumors (see middle panel of FIG. 26 for histology). The vascular space of both bronchi was perfused for 28 days through the pulmonary artery. Circulating cells were observed starting at day 4-5, and the number of circulating cells increased over time (see lower panel of FIG. 26).

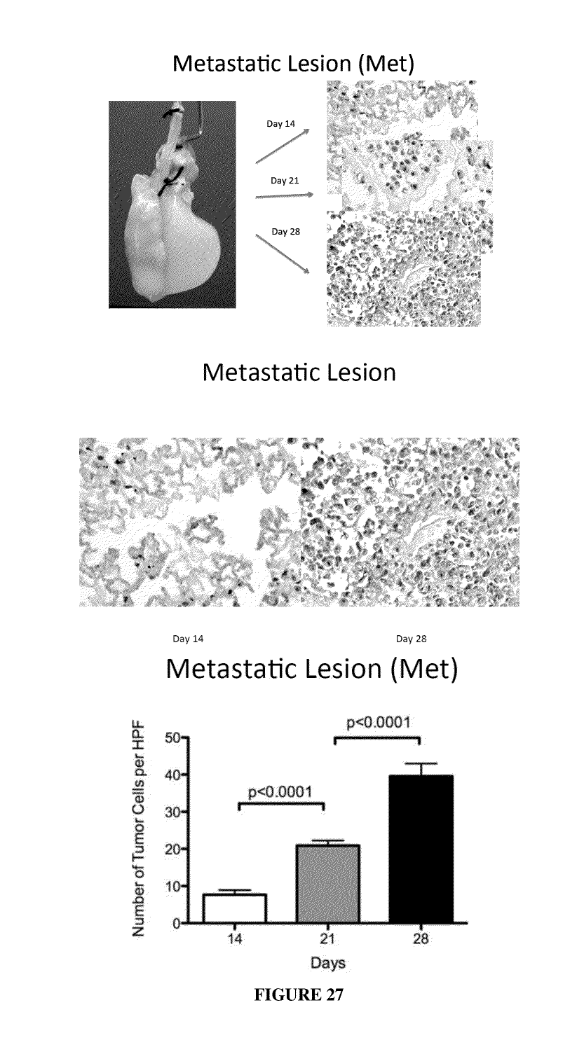

In order investigate whether circulating cells originating from a primary tumor in the open bronchus were able to reach the interstitial space of the tied-off bronchus and form secondary tumors there, lobectomy of the tied-off bronchus was performed at day 14 (upper lobe), day 21 (middle lobe), and day 28 (lower lobe), and the histology of the excised lobes was evaluated (FIG. 27, upper panel). It was observed that the excised lobes contained tumor nodules, which increased in number and size over time (FIG. 27, middle and lower panels). This observation is consistent with the notion that cells from the primary tumor nodules in the open bronchus undergo EMT and transgress the basement membrane of the open bronchus to form circulating cells. Some of the circulating cells then transgress the basement membrane of the tied-off bronchus and populate the interstitial space of that bronchus, forming secondary nodules, which represent metastatic lesions. Lower panel: number of circulating cells measured over a time period of 28 days in the metastatic model.

### Discussion

Native lung extracellular biomatrix is a complex system that provides support to normal tissue and maintains cell-cell interactions, cell-matrix interactions, cellular differentiation, and tissue organization. Several groups have been successful recently in creating a pure biomatrix using cadaveric lung [9, 13-20]. Some have been successful in populating the decellularized organ with normal cells to recreate an organ for transplantation [9, 13, 15]. The bioreactor needed to develop an organ suitable for orthotopic transplantation into a rat is complex, requiring precise control of flow and pressure through the circulation and a ventilation loop through the trachea. Because our goal was to develop a three-dimensional lung cancer cell culture model system, this complexity was not needed.

A simpler bioreactor was created from existing parts that has a pump and oxygenator without a ventilator loop, and found that it was adequate for growing human lung cancer cell lines to form perfusable lung cancer nodules with features similar to the original human lung cancer.

The A549 cells (derived from human bronchial adenocarcinoma) formed nodules with cancer cells in a lepidic growth pattern characterized by tumor cells growing along preexisting alveolar structures. The tumor cells were organized to show cell-cell interaction and cell-biomatrix interaction, suggested by the immunohistochemistry staining. The nuclei were oval-round with prominent nucleoli, typical of moderately differentiated and well-differentiated adenocarcinoma. The cells lacked a desmoplastic stromal reaction typically seen in human lung cancer, likely attributable to a lack of mesenchymal cells. When the H460 cells (derived from pleural fluid cells of a patient with large cell lung cancer) were placed on the scaffold, they formed vascularized lung nodules like the A549 cells grown on the biomatrix but the pathologic appearance was similar to human large cell lung cancer.

Finally, when the H1299 cells (derived from the metastatic lymph node from a patient with non-small cell lung cancer) were placed on the scaffold, the vascularized tumor nodules formed on the biomatrix like the other two cell types, but the pathologic appearance was similar to tumor growth in a lymph node. These results suggest that the cancer cells retain the necessary information to form complex nodules similar to the original cancer cells.

This new ex vivo system is a significant addition to the in vitro and in vivo model systems currently available to study human lung cancer. To date, there is no system that can create perfusable lung cancer nodules with the histopathologic features of lung tumors that are similar morphologically to the original human lung cancer. The cells grow into a complex structure that is not seen in simple monolayer cell cultures. Moreover, as the tumor grows, it develops characteristic features of mitosis and apoptosis, which are difficult to appreciate with other in vitro models.

This work provides a new tool for studying lung cancer in an ex vivo environment that closely simulates the actual tumor microenvironment, having essential characteristics such as colocalization of different cell types with cell-cell interactions in the presence of extracellular biomatrix to provide a scaffold for mechanical stability and to regulate cell function [21]. This model can be used to improve our understanding of the tumor microenvironment and angiogenesis in lung tumor development.

Using a simple decellularization chamber and bioreactor, aspects of this disclosure show that human neoplastic cells, for example, lung cancer cell lines, can be cultured within a decellularized rat lung biomatrix in a manner that mimics cancer, e.g., human lung cancer.

### REFERENCES

1 Jemal A, Siegel R, Xu J, Ward E. Cancer statistics, 2010. CA Cancer J Clin 2010; 60:277 300.
2 Staton C A, Stribbling S M, Tazzyman S, Hughes R, Brown N J, Lewis C E. Current methods for assaying angiogenesis in vitro and in vivo. Int J Exp Pathol 2004; 85:233-48.
3 Kleinman H K, Jacob K. Invasion assays. In: Bonifacino J S, et al, eds. Current protocols in cell biology/editorial board, Chapter 12.2001:Unit 12 12.
4 Albini A, Noonan D M. The ‘chemoinvasion’ assay, 25 years and still going strong: the use of reconstituted basement membranes to study cell invasion and angiogenesis. Curr Opin Cell Biol 2010; 22:677-89.
5 Rintoul R C, Sethi T. The role of extracellular matrix in small-cell lung cancer. Lancet Oncol 2001; 2:437-42.
6 Petersen O W, Rønnov-Jessen L, Howlett A R, Bissell M J. Interaction with basement membrane serves to rapidly distinguish growth and differentiation pattern of normal and malignant human breast epithelial cells. Proc Natl Acad Sci USA 1992; 89:9064-8.
7 Bergstraesser L M, Weitzman S A. Culture of normal and malignant primary human mammary epithelial cells in a physiological manner simulates in vivo growth patterns and allows discrimination of cell type. Cancer Res 1993; 53:2644-54.
8 Kunz-Schughart L A, Heyder P, Schroeder J, Knuechel R. A heterologous 3-D coculture model of breast tumor cells and fibroblasts to study tumor-associated fibroblast differentiation. Exp Cell Res 2001; 266:74-86.
9 Ott H C, Clippinger B, Conrad C, et al. Regeneration and orthotopic transplantation of a bioartificial lung. Nat Med 2010; 16:927-33.
10 Uygun B E, Soto-Gutierrez A, Yagi H, et al. Organ reengineering through development of a transplantable recellularized liver graft using decellularized liver matrix. Nat Med 2010; 16:814-20.
11 Kuttan R, Spall R D, Duhamel R C, Sipes I G, Meezan E, Brendel K. Preparation and composition of alveolar extracellular matrix and incorporated basement membrane. Lung 1981; 159:333-45.
12 Kiernan J, ed. Histological and histochemical methods: theory and practice, 4th ed. Bloxham, UK: Scion Publishing, 2008.
13 Song J J, Kim S S, Liu Z, et al. Enhanced in vivo function of bioartificial lungs in rats. Ann Thorac Surg 2011; 92:998-1006.
14 Shamis Y, Hasson E, Soroker A, Bassat E, Shimoni Y, et al. Organ-specific scaffolds for in vitro expansion, differentiation, and organization of primary lung cells. Tissue engineering Part C, Methods 2011; 17:861-70.
15 Petersen T H, Calle E A, Colehour M B, Niklason L E. Matrix composition and mechanics of decellularized lung scaffolds. Cells Tissues Organs 2011 Apr. 18. [Epub ahead of print].
16 Kubo H. Molecular basis of lung tissue regeneration. Gen Thorac Cardiovasc Surg 2011; 59:231-44.
17 Shamis Y, Hasson E, Soroker A, et al. Organ-specific scaffolds for in vitro expansion, differentiation and organization of primary lung cells. Tissue Eng Part C Methods 2011; 17: 861-70.
18 Soucy P A, Werbin J, Heinz W, Hoh J H, Romer L H. Microelastic properties of lung cell-derived extracellular matrix. Acta Biomater 2011; 7:96-105.
19 Price A P, England K A, Matson A M, Blazar B R, Panoskaltsis-Mortari A. Development of a decellularized lung bioreactor system for bioengineering the lung: the matrix reloaded. Tissue Eng Part A 2010; 16:2581-91.
20 Nichols J E, Niles J A, Cortiella J. Design and development of tissue lung: progress and challenges. Organogenesis 2009; 5:57-61.
21 Kim J B. Three-dimensional tissue culture models in cancer biology. Semin Cancer Biol 2005; 15:365-77.

All publications, patents and database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a cell” or “the cell” also includes the plural forms “cells” or “the cells,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A tissue construct, comprising
a decellularized biomatrix; and
a neoplastic cell cultured within the decellularized biomatrix.

2. The tissue construct of claim 1, wherein the tissue construct comprises a tumor nodule.

3. The tissue construct of claim 1, wherein the neoplastic cell is not native to the decellularized biomatrix.

4. The tissue construct of claim 1, wherein the neoplastic cell is from a different species than the decellularized biomatrix.

5. The tissue construct of claim 1, wherein the decellularized biomatrix is derived from a healthy tissue or organ obtained from a subject.

6. The tissue construct of claim 1, wherein the tissue construct comprises a perfusable vasculature.

7. The tissue construct of claim 1, wherein the decellularized biomatrix comprises lung biomatrix.

8. The tissue construct of claim 1, wherein the decellularized biomatrix comprises rat or mouse biomatrix.

9. The tissue construct of claim 1, wherein the neoplastic cell is a human cell.

10. The tissue construct of claim 1, wherein the neoplastic cell is a tumor or cancer cell.

11. The tissue construct of claim 1, wherein the tissue construct further comprises a non-neoplastic cell.

12. A method for preparing a tissue construct, comprising
providing a decellularized biomatrix; and
contacting the decellularized biomatrix with a neoplastic cell under conditions suitable for the neoplastic cell to grow within the decellularized biomatrix.

13.-20. (canceled)

21. The method of claim 12, wherein the method further comprises analyzing the tissue construct.

22. The method of claim 21, wherein the analyzing comprises observing a tumor nodule, observing growth of a tumor nodule, quantifying a number of tumor nodules, assaying expression of a gene product associated with neoplasia, assaying cell survival or cell death, assaying metastatic potential, and/or assaying a signaling factor associated with neoplasia.

23. A method of identifying an anti-cancer agent, the method comprising
(a) contacting the tissue construct of claim 1 with a candidate agent;
(b) assessing a biomarker associated with cancer in the tissue construct contacted with the candidate agent; and
(c) comparing the assessed biomarker of (b) with a reference value;
wherein, if the biomarker associated with cancer is absent or diminished in the tissue construct contacted with the candidate agent as compared to the reference value, then the candidate agent is identified as an anti-cancer agent.

24. The method of claim 23, wherein the biomarker assessed in (b) comprises cell proliferation, cell survival, tumor formation, tumor number, tumor growth, tumor volume, tumor phenotype, tumor nodule formation, tumor nodule number, tumor nodule growth, tumor nodule structure, tumor nodule volume, tumor nodule phenotype, expression of a gene product, expression of an oncogene, repression of a tumor suppressor, presence or abundance of neoplastic cells in a perfusion efflux fluid, expression of mesenchymal markers by cells present in a perfusion fluid, and/or a metastatic activity of cells present in a perfusion fluid.

25.-27. (canceled)

28. A bioreactor for growing perfusable tissue constructs, the bioreactor comprising
a decellularized biomatrix comprising a vascular space and an epithelial space;
a perfusion influx connected to the vascular space;
a perfusion efflux connected to the vascular space;
a culture media influx connected to the epithelial space; and
a neoplastic cell growing within the decellularized biomatrix and contacted with the culture media.

29.-33. (canceled)

34. A metastatic tumor model comprising a decellularized biomatrix, the decellularized biomatrix comprising:
a primary interstitial space and a neoplastic cell within the primary interstitial space;
a secondary interstitial space, wherein the secondary interstitial space does not comprise a neoplastic cell;
a barrier to cell migration that separates the primary and the secondary interstitial space; and
a vascular space shared by the primary interstitial space and the secondary interstitial space, wherein the vascular space comprises a perfusion medium.

35.-39. (canceled)

40. A method for cultivating neoplastic cells, the method comprising providing a decellularized biomatrix comprising
a primary interstitial space;
a secondary interstitial space, wherein the secondary interstitial space does not comprise a neoplastic cell;
a barrier to cell migration that separates the primary and the secondary interstitial space; and
a vascular space shared by the primary interstitial space and the secondary interstitial space, wherein the vascular space comprises a perfusion medium; and
contacting the primary interstitial space of the biomatrix with a neoplastic cell under conditions suitable for the neoplastic cell to grow within the decellularized biomatrix.

41.-48. (canceled)

49. A method of identifying an anti-metastatic agent, the method comprising
(a) contacting the metastatic tumor model of claim 34 with a candidate agent;
(b) assessing a biomarker associated with metastasis in the tissue construct contacted with the candidate agent; and
(c) comparing the assessed biomarker of (b) with a reference value;
wherein, if the biomarker associated with metastasis is absent or diminished in the tissue construct contacted with the candidate agent as compared to the reference value, then the candidate agent is identified as an anti-metastatic agent.

50-53. (canceled)
